(19) United States
(12) Patent Application Publication
(10) Pub. No.: US 2012/0329832 A1
(43) Pub. Date: Dec. 27, 2012

(54) Novel Insect-Repellent Coumarin Derivatives, Syntheses, and Methods of Use

(76) Inventors: Jean Delaveau, Saint Nicolas de Macherin (FR); Emile Vialle, Beauregard L'Eveque (FR); Marc Lemaire, Villeurbanne (FR); Stephane Pellet-Rostaing, Villeurbanne (FR); Bruno Andrioletti, Chemin de Rosarge (FR)

(21) Appl. No.: 13/528,944

(22) Filed: Jun. 21, 2012

Related U.S. Application Data
(60) Provisional application No. 61/501,485, filed on Jun. 27, 2011.

(51) Int. Cl. A01N 43/40; A01N 43/16; C07D 311/22; C07D 311/14; A61Q 17/02; C07D 405/06; C07C 69/732; A01N 37/10; A61K 8/49; A01P 17/00; C07D 311/16; C07D 407/12

(52) U.S. Cl. 514/320; 549/289; 514/457; 549/285; 546/196; 560/75; 514/532; 549/283

(57) ABSTRACT

This invention relates to novel coumarin derivative, formulations comprising same, and to methods of making and using these compounds and formulations, which are useful as repellents against insects and/or pests. The compounds also prevent illness and disease caused by insect/pest-borne vectors, and provide safer, more effective alternatives to existing repellents.

### INCORPORATION BY REFERENCE

This application claims benefit of the U.S. provisional patent application Ser. No. 61/501,485 filed on Jun. 27, 2011, and herein incorporated by reference in its entirety.

### FIELD OF THE INVENTION

The present invention relates to novel coumarin derivatives, a method for making such compounds, and a method of repelling insects and pests away from animals, including humans. The present invention has particular, though not sole, application to repelling insects including flies and mosquitoes.

### BACKGROUND

Repellents are used to keep pests, including insects and acarids, away from animals including humans. In addition to being a nuisance, pests are vectors for many disease/disorder-causing agents including parasites, bacteria, and virus. Several known repellents are presented in Table 1.

TABLE 1
Commonly known repellents

| | | Mole- | |
|---|---|---|---|
| | | cular | |
| Name | Molecule | mass | Formula |
| Diethyl- tolumide (DEET) | 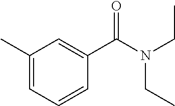 | 191.13 | C₁₂H₁₇NO |
| Picaridine | 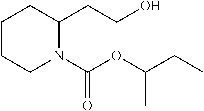 | 229.17 | C₁₂H₂₃NO₃ |
| Benzyl benzoate | 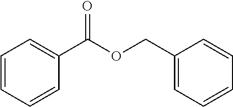 | 212.08 | C₁₄H₁₂O₂ |
| Coumarin | 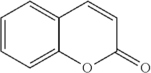 | 146.15 | C₉H₆O₂ |

N,N-Diethyl-meta-toluamide, abbreviated DEET, is a slightly yellow oil. It is the most common active ingredient in insect repellents. It is intended to be applied to the skin or to clothing, and is primarily used to repel mosquitoes. In particular, DEET is known to provide some protection against tick bites and mosquito bites, which can both transmit disease. DEET was historically believed to work by blocking insect olfactory receptors for 1-octen-3-ol, a volatile substance that is contained in human sweat and breath. The prevailing theory was that DEET effectively “blinded” the insect's senses so that the biting/feeding instinct is not triggered by humans or other animals which produce these chemicals. DEET does not appear to affect the insect's ability to smell carbon dioxide, as had been suspected earlier (Petherick et al., 2008; Ditzen et al., 2008) However, more recent evidence shows that DEET serves as a true repellent in that mosquitoes intensely dislike the smell of the chemical repellent (Syed and Leal, 2008). A type of olfactory receptor neuron in special antennal sensilla of mosquitoes that is activated by DEET as well as other known insect repellents such as eucalyptol, linalool, and thujone has been identified. Moreover, in a behavioral test DEET had a strong repellent activity in the absence of body odor attractants such as 1-octen-3-ol, lactic acid, or carbon dioxide.

DEET health effects. As a precaution, manufacturers advise that DEET products should not be used under clothing or on damaged skin, and that preparations be washed off after they are no longer needed or between applications. As a precaution, manufacturers advise that DEET products should not be used under clothing or on damaged skin, and that preparations be washed off after they are no longer needed or between applications. DEET can act as an irritant; in rare cases, it may cause skin reactions (CDC Report). In the DEET Reregistration Eligibility Decision (RED), the US Environmental Protection Agency (EPA) reported 14 to 46 cases of potential DEET-associated seizures, including 4 deaths. The EPA states: “ . . . it does appear that some cases are likely related to DEET toxicity,” but observed that with 30% of the US population using DEET, the likely seizure rate is only about one per 100 million users (US EPA, 1998). The Pesticide Information Project of Cooperative Extension Offices of Cornell University states that “Everglades National Park employees having extensive DEET exposure were more likely to have insomnia, mood disturbances and impaired cognitive function than were lesser exposed co-workers” (Extoxnet, 1997). When used as directed, products containing between 10% to 30% DEET have been found by The American Academy of Pediatrics to be safe to use on children as well as adults but recommends that DEET not be used on infants less than two months old (CDC report). Citing human health reasons, Health Canada barred the sale of insect repellents for human use that contained more than 30% DEET in a 2002 re-evaluation. The agency recommended that DEET based products be used on children between the ages of 2 and 12 only if the concentration of DEET is 10% or less and that repellents be applied no more than 3 times a day, children under 2 should not receive more than 1 application of repellent in a day and DEET based products of any concentration not be used on infants under 6 months (Health Canada, 2009). Recently, DEET has been found to inhibit the activity of a central nervous system enzyme, acetylcholinesterase, in both insects and mammals (Corbel et al., 2009). This enzyme is involved in the hydrolysis of the neurotransmitter acetylcholine, thus playing a role in the function of the neurons which control muscles. Because of this property, many insecticides are used to block acetylcholinesterase, which leads to an excessive accumulation of acetylcholine at the synaptic cleft, causing neuromuscular paralysis and death by asphyxiation (Purves et al., 2008). DEET is commonly used in combination with insecticides and has the capacity to strengthen the toxicity of carbamates (Moss, 1996), a class of insecticides known to block acetylcholinesterase. These findings bring evidence that, besides having known toxic effects on the olfactory system, DEET also acts on the brain of insects, and that its toxicity is strengthened in combination with other insecticides.

Icaridin, also known as picaridine, KBR 3023, under the INCI name hydroxyethyl isobutyl piperidine carboxylate, and the trade names BAYREPEL and SALTIDIN, is an insect repellent. It has broad efficacy against different insects and is almost colorless and odorless. Icaridin has been reported to be as effective as DEET without the irritation associated with DEET (J. of Drugs and Dermatology, January-February 2004). According to the WHO, icaridin “demonstrates excellent repellent properties comparable to, and often superior to, those of the standard DEET.” Picaridin, first used in Europe in 2001, has been reported to be effective by Consumer Reports (7% solution) and the Australian Army (20% solution). Consumer Reports retests in 2006 gave as result that a 7% solution of picaridin offered little or no protection against Aedes mosquitoes (vector of dengue fever) and a protection time of about 2.5 hours against Culex (vector of West Nile virus), while a 15% solution was good for about one hour against Aedes and 4.8 hours against Culex (Consumer Reports, June 2006, pg. 6).

Coumarin (2H-chromen-2-one) is a pleasantly fragrant benzopyrone/phenylpropanoid chemical compound found in many plants, notably in high concentration in the tonka bean (Dipteryx odorata), vanilla grass (Anthoxanthum odoratum), sweet woodruff (Galium odoratum), mullein (Verbascum spp.), sweet grass (Hierochloe odorata), Cassia cinnamon (Cinnamomum aromaticum) and sweet clover. The name comes from a French word, coumarou, for the tonka bean.

There are clear, long-felt, and unmet needs for new repellents having improved safety and efficacy profiles. It is therefore an object of the instant invention to provide novel derivatives of coumarin having improved safety and efficacy as compared with currently available repellents.

It is expressly noted that citation or identification of any document in this application is not an admission that such document is available as prior art to the present invention. Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### SUMMARY OF THE INVENTION

A first aspect of the invention is to provide novel compounds that are based upon the bicyclic compound coumarin, and are active in repelling pests, including insects and acarids, that are a burden to animals including humans. In some embodiments, the compounds are effective repellents against mosquitoes, flies, ticks, and fleas.

The invention is also directed toward a method of protecting an animal (e.g. a mammal or bird) against pests by administering a effective repelling amount of the compositions of the invention. Animals which can be treated include but are not limited to chickens/avians, humans, cats, dogs, cattle, cows, deer, goats, horses, llamas, pigs, sheep and yaks. In one embodiment of the invention, the animals treated are canines, felines, or humans.

In one embodiment, the present invention provides coumarin derivative compounds of formula (I) or (Ia) shown below:

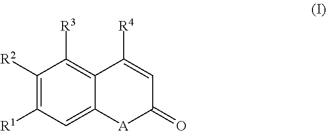

or a veterinarily or pharmaceutically acceptable salt thereof; wherein R¹, R², R³, and R⁴ independently include H, C, OR⁵, CR⁵, OCC(═O)N(R⁵)(R⁷), CC(═O)N(R⁵)(R⁷), and wherein A is O, or the ring is opened at A, thus forming compounds according to formula (Ia):

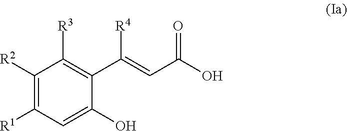

and wherein additional meanings of variables R¹, R², R³, R⁴, R⁵, R⁶ and R⁷ are as described below. The invention also provides veterinary and pharmaceutical compositions comprising the inventive compounds, or salts thereof, in combination with a veterinarily or pharmaceutically acceptable carrier or diluent.

The inventive compounds and compositions comprising the compounds are highly effective for the repulsion of pests. Accordingly, the present invention provides methods for repelling pests away from animals, including humans, comprising applying a repulsive effective amount of a compound of formula (I), or a veterinarily or pharmaceutically acceptable salt thereof, to the animal or human, or its surroundings.

It is an object of the invention to not encompass within the invention any previously known product, process of making the product, or method of using the product such that the Applicants reserve the right to this invention and hereby disclose a disclaimer of any previously known product, process, or method.

It is noted that in this disclosure and particularly in the claims, terms such as “comprises”, “comprised”, “comprising” and the like can have the meaning attributed to such terms in U.S. patent law; e.g., they can mean “includes”, “included”, “including”, and the like; and that terms such as “consisting essentially of” and “consists essentially of” have the meaning ascribed to them by U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF DRAWINGS

A full and enabling disclosure of the present invention, including the best mode thereof, to one of ordinary skill in the art, is set forth more particularly in the remainder of the specification, including reference to the accompanying figures, wherein:

FIG. 1 is a graph indicating Repulsive Index (RI) for solutions of DEET, picaridine, EV06166, and EV06718;

FIG. 2 is a graph indicating RI for 10% and 20% solutions of EV05120 and EV06096;

FIG. 3 is a graph indicating RI for 10% and 20% solutions of EV05174 and EV05178;

FIG. 4 is a graph indicating RI for DEET, picaridine, and eight coumarin derivatives according to the instant disclosure;

FIG. 5 is a graph indicating RI for DEET, picaridine, six C4-substituted, and three C6-substituted coumarin derivatives according to the instant disclosure;

FIG. 6 is a graph indicating RI for DEET, EV06148, EV06144, EV06044, EV06110, in solutions with concentrations varying from 1% to 20%;

FIG. 7 is a graph indicating RI for DEET, EV06062, EV06098, and EV06088 in solutions with concentrations varying from 1% to 20%;

FIG. 8 is a graph indicating RI for DEET, EV06068, and EV06154 in solutions with concentrations varying from 1% to 20%;

FIG. 9 is a graph indicating RI for EV04036, EV06026, EV06110, and EV06144;

FIG. 10 is a graph indicating RI for C7-substituted ethers containing 5 carbons;

FIG. 11 is a graph indicating RI for C4-substituted ethers containing 5 carbons;

FIG. 12 is a graph indicating RI for 3-pentanol-substituted derivatives;

FIG. 13 is a graph indicating RI for (S)-Butan-2-ol-substituted derivatives;

FIG. 14 is a graph indicating RI for 2-pentanol-substituted derivatives;

FIG. 15 is a graph indicating RI for R, S, or R/S Butan-2-ol-substituted derivatives;

FIG. 16 is a graph indicating RI for DEET, picaridine, EV06062, EV06028, EV05120, and EV05174;

FIG. 17 is a graph indicating the R1 for DEET, picaridine, and twenty-two novel coumarin derivatives;

FIG. 18 is a graph indicating the R1 for DEET, picaridine, and eleven novel coumarin derivatives;

FIG. 19 is a graph indicating RI versus concentration for DEET, EV06062, EV06148, EV06096, EV05174, and EV05178;

FIG. 20 is a graph indicating RI for selected inventive compounds (and DEET, picaridin). As shown, several compounds have RI≧RI for DEET.

### DETAILED DESCRIPTION OF THE INVENTION

The present invention provides novel coumarin derivative compounds with insect and pest repellent activity, or pharmaceutically/veterinarily acceptable or pharmaceutically acceptable salts thereof, and compositions comprising the compounds or salts for the repulsion of insects or other pests away from an animal or a human. An important aspect of the invention is to provide coumarin derivative compounds with high repellent activity against pests, particularly though not solely insects, and improved safety to the user, the environment, and the animal.

The invention includes at least the following features:

(a) In one embodiment, the invention provides novel compounds of formula (I) and (II), or veterinarily or pharmaceutically acceptable salts thereof, which is a repellent of animal pests, including insects and acarids;

(b) veterinary and pharmaceutical compositions for repelling pests comprising repellent effective amount of the compounds of formula (I), or veterinarily or pharmaceutically acceptable salts thereof, in combination with a veterinarily or pharmaceutically acceptable carrier or diluent;

(c) veterinary and pharmaceutical compositions for repelling pests comprising a repellent effective amount of the compounds of the invention, or veterinarily or pharmaceutically acceptable salts thereof, in combination with one more other active agent, including other repellents, antiparasitics, and a veterinarily or pharmaceutically acceptable carrier or diluent;

(d) methods for repelling pests, including insects and acarids, away from an animal, including a human, are provided, which methods comprise administering a repellent effective amount of a compound of formula (I), or veterinarily acceptable salts thereof, to the animal in need thereof;

(e) methods for the prevention of infestation/infection and/or the reduction of transmission of a pest-borne pathogen to animals, including humans, which comprise administering a repellent effective amount of a compound of formula (I), or veterinarily or pharmaceutically acceptable salts thereof, to the animal in need thereof, thereby preventing infection/infestation and/or reducing the transmission of pest-borne pathogens to animals, including humans;

(f) methods for controlling pests at a locus (e.g. by repelling pests away from a locus), comprising administering or applying a repellent effective amount of a compound of formula (I), or veterinarily or pharmaceutically acceptable salts thereof, to the locus;

(g) use of the compounds of formula (I), or veterinarily acceptable salts thereof, in the manufacture of a veterinary or pharmaceutical medicament for repelling pests, including insects and acarids; and

(h) processes for the preparation of the compounds of formula (I).

### DEFINITIONS

Terms used herein will have their customary meanings in the art unless specified. The organic moieties mentioned in the definitions of the variables of formula (I) are like the term halogen—i.e., collective terms for individual listings of the individual group members. The prefix Cₙ-Cₘ indicates in each case the possible number of carbon atoms in the group.

The term “alkyl” refers to saturated straight, branched, cyclic, primary, secondary or tertiary hydrocarbons, including those having 1 to 12 atoms. In some embodiments, alkyl groups will include C₁-C₁₀, C₁-C₈, C₁-C₆ or C₁-C₄ alkyl groups. Examples of C₁-C₁₀ alkyl include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

Cyclic alkyl groups, which are encompassed by alkyls, may be referred to as “cycloalkyl” and include those with 3 to 10 carbon atoms having single or multiple fused rings. Non-limiting examples of cycloalkyl groups include adamantyl, cyclopropyl, cyclobutyl, cyclopentyl, cyclohexyl, cycloheptyl, cyclooctyl and the like.

The alkyl and cycloalkyl groups described herein can be unsubstituted or substituted with one or more moieties selected from the group consisting of alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the biological activity of the compounds of the invention, either unprotected, or protected as necessary, as known to those skilled in the art, for example, as taught in Greene, et al., Protective Groups in Organic Synthesis, John Wiley and Sons, Third Edition, 1999, hereby incorporated by reference.

The term “alkenyl” refers to both straight and branched carbon chains which have at least one carbon-carbon double bond. In some embodiments, alkenyl groups may include C₂-C₁₂ alkenyl groups. In other embodiments, alkenyl includes C₂-C₁₀, C₂-C₈, C₂-C₆ or C₂-C₄ alkenyl groups. In one embodiment of alkenyl, the number of double bonds is 1-3; in another embodiment of alkenyl, the number of double bonds is one. Other ranges of carbon-carbon double bonds and carbon numbers are also contemplated depending on the location of the alkenyl moiety on the molecule. “C₂-C₁₀-alkenyl” groups may include more than one double bond in the chain. Examples include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

“Cycloalkenyl” refers to monovalent cyclic alkenyl groups of from 4 to 10 carbon atoms, preferably 5 to 8 carbon atoms, having single or multiple fused rings which fused rings may or may not be cycloalkenyl provided that the point of attachment is to a cycloalkenyl ring atom. Examples of cycloalkenyl groups include, by way of example, cyclopenten-4-yl, cyclooctene-5-yl and the like. Alkenyl and cycloalkenyl groups may be unsubstituted or substituted with one or more substituents as described for alkyl above.

“Alkynyl” refers to both straight and branched carbon chains which have at least one carbon-carbon triple bond. In one embodiment of alkynyl, the number of triple bonds is 1-3; in another embodiment of alkynyl, the number of triple bonds is one. In some embodiments, alkynyl groups include from 2 to 12 carbon atoms. In other embodiments, alkynyl groups may include C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term “haloalkyl” refers to an alkyl group, as defined herein, which is substituted by one or more halogen atoms. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “fluoroalkyl” as used herein refers to an alkyl in which one or more of the hydrogen atoms is replaced with fluorine atoms, for example difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “haloalkenyl” refers to an alkenyl group, as defined herein, which is substituted by one or more halogen atoms.

The term “haloalkynyl” refers to an alkynyl group, as defined herein, which is substituted by one or more halogen atoms.

“Alkoxy” refers to alkyl-O—, wherein alkyl is as defined above. Similarly, the terms “alkenyloxy,” “alkynyloxy,” “haloalkoxy,” “haloalkenyloxy,” “haloalkynyloxy,” “cycloalkoxy,” “cycloalkenyloxy,” “halocycloalkoxy,” and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined above. Examples of C₁-C₆-alkoxy include, but are not limited to, methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

“Aryl” refers to a monovalent aromatic carbocyclic group of from 6 to 14 carbon atoms having a single ring or multiple fused rings. Aryl groups include, but are not limited to, phenyl, biphenyl, and naphthyl. In some embodiments aryl includes tetrahydronapthyl, phenylcyclopropyl and indanyl. Aryl groups may be unsubstituted or substituted by one or more moieties selected from halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenylsulfonyl, haloalkynylsulfonyl, alkylamino, alkenylamino, alkynylamino, di(alkyl)amino, di(alkenyl)-amino, di(alkynyl)amino, or trialkylsilyl.

The term “aralkyl” refers to an aryl group that is bonded to the parent compound through a diradical alkylene bridge, (—CH₂—)ₙ, where n is 1-12 and where “aryl” is as defined above.

“Heteroaryl” refers to a monovalent aromatic group of from 1 to 15 carbon atoms, preferably from 1 to 10 carbon atoms, having one or more oxygen, nitrogen, and sulfur heteroatoms within the ring, preferably 1 to 4 heteroatoms, or 1 to 3 heteroatoms. The nitrogen and sulfur heteroatoms may optionally be oxidized. Such heteroaryl groups can have a single ring (e.g., pyridyl or furyl) or multiple fused rings provided that the point of attachment is through a heteroaryl ring atom. Examples of heteroaryls include pyridyl, piridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings may be unsubstituted or substituted by one or more moieties as described for aryl above.

“Heterocyclyl,” “heterocyclic” or “heterocyclo” refers to fully saturated or unsaturated, cyclic groups, for example, 3 to 7 membered monocyclic, 7 to 11 membered bicyclic, or 10 to 15 membered tricyclic ring systems, which have one or more oxygen, sulfur or nitrogen heteroatoms in ring, preferably 1 to 4 or 1 to 3 heteroatoms. The nitrogen and sulfur heteroatoms may optionally be oxidized and the nitrogen heteroatoms may optionally be quaternized. The heterocyclic group may be attached at any heteroatom or carbon atom of the ring or ring system and may be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

Exemplary bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), tetrahydroquinolinyl and the like.

The term “alkylthio” or “alkylsulfanyl” refers to alkyl-S—, where “alkyl” is as defined above. In some embodiments, the alkyl component of the alkylthio group will include C₁-C₁₀, C₁-C₈, C₁-C₆ or C₁-C₄ alkyl groups. For example, C₁-C₄-alkylthio include, but are not limited to, methylthio, ethylthio, propylthio, 1-methylethylthio, butylthio, 1-methylpropylthio, 2-methylpropylthio or 1,1-dimethylethylthio. Similarly, the terms “haloalkylthio,” “cycloalkylthio,” “halocycloalkylthio” refer to the groups —S-haloalkyl, —S-cycloalkyl, and —S-halocycloalkyl, respectively, where the terms “haloalkyl,” “cycloalkyl,” and “halocycloalkyl” are as defined above.

The term “alkylsulfinyl” refers to the group alkyl-S(═O)—, where “alkyl” is as defined above. In some embodiments, the alkyl component in alkylsulfinyl groups will include C₁-C₁₂, C₁-C₁₀, C₁-C₈, C₁-C₆ or C₁-C₄ alkyl groups. Examples include, but are not limited to, —SO—CH₃, —SO—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

Similarly, the terms “alkenylfulfinyl,” “alkynylsulfinyl,” “haloalkylsulfinyl,” “haloalkenylsulfinyl,” and “haloalkynylsulfinyl” refer to the groups alkenyl-S(═O)—, alkynyl-S(═O)—, and haloalkyl-S(═O)—, haloalkenyl-S(═O)—, and haloalkynyl-S(═O)—, where the terms “alkenyl,” “alkynyl,” “haloalkyl,” “haloalkenyl,” and “haloalkynyl” are as defined above. The term “alkylsulfonyl” refers to the group alkyl-S(═O)₂—, where the term “alkyl” is as defined above. In some embodiments, the alkyl component in alkylsulfonyl groups will include C₁-C₁₂, C₁-C₁₀, C₁-C₈, C₁-C₆ or C₁-C₄ alkyl groups. Examples include, but are not limited to, —SO₂—CH₃, —SO₂—C₂H₅, n-propylsulfonyl, —SO₂—CH(CH₃)₂, n-butylsulfonyl, 1-methylpropylsulfonyl, 2-methylpropylsulfonyl, —SO₂—C(CH₃)₃, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl and the like.

The terms “alkenylfulfonyl,” “alkynylsulfonyl,” “halo alkylsulfonyl,” “haloalkenylsulfonyl,” and “haloalkynylsulfonyl” refer to the groups alkenyl-S(═O)₂—, alkynyl-S(═O)₂—, and haloalkyl-S(═O)₂—, haloalkenyl-S(═O)₂—, and haloalkynyl-S(═O)₂—, where the terms “alkenyl,” “alkynyl,” “haloalkyl,” “haloalkenyl,” and “haloalkynyl” are as defined above. The terms “alkylamino,” “dialkylamino,” “alkenylamino,” “alkynylamino,” “di(alkenyl)amino,” and “di(alkynyl)amino” refer to the groups —NH(alkyl), —N(alkyl)₂, —NH(alkenyl), —NH(alkynyl), —N(alkenyl)₂ and —N(alkynyl)₂, where the terms “alkyl,” “alkenyl,” and “alkynyl” are as defined above. In some embodiments, the alkyl component in alkylamino or dialkylamino groups will include C₁-C₁₂, C₁-C₁₀, C₁-C₈, C₁-C₆ or C₁-C₄ alkyl groups.

The term “trialkylsilyl” refers to the group —Si(alkyl)₃, where the group is bonded to the parent compound at the silicon atom.

### Compounds of the Invention:

In certain embodiments, the compounds of the invention are useful in veterinary applications, including for repelling pests, including insects and acarids, away from an animal. In other embodiments, the inventive compounds are useful in pharmaceutical or veterinary applications for repelling insects or acarids.

In one embodiment the invention provides a coumarin derivative of formula (I), or a veterinarily or pharmaceutically acceptable salt thereof:

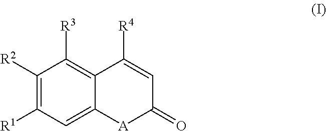

wherein

R¹, R², R³, and R⁴ independently include H, C, OR⁵, CR⁵, OCC(═O)N(R⁵)(R⁷), CC(═O)N(R⁵)(R⁷), alkyl, aryl, aralkyl, heteroaryl, alcohols, amine, aldehyde, heterocyclyl, or salts of amines and carboxylates; wherein A is 0, or the ring is opened at A, thus forming compounds according to formula (Ia):

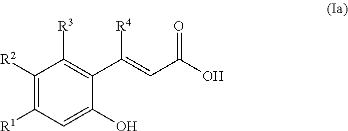

In an embodiment of the invention the compound of formula (I) or (Ia) is selected from the compounds described in Tables 2-17.

In another embodiment, the compound of formula (I) is EV04016, EV04024, EV04030, EV04032, EV04036, EV04054, EV04058, EV04062, EV04070, EV04084, EV04090, EV04094, EV04114, EV04122, EV04188, EV05056, EV05084, EV05088, EV05096, EV05118, EV05120, EV05134, EV05138, EV05144, EV05162, EV05174, EV05178, EV05184, EV06018, EV06020, EV06026, EV06028, EV06036, EV06044, EV06046, EV06048, EV06056, EV06058, EV06062, EV06068, EV06072, EV06086, EV06088, EV06092, EV06094, EV06096, EV06098, EV06110, EV06128, EV06136, EV06140, EV06144, EV06148, EV06154, EV06162, EV06166, EV06178, EV06188, EV07038.

In another embodiment, the compound of formula (I) is set forth according to the following provisions:

A=O

R²=R³=R⁴ is H;

R¹ is OC(═O)OR⁵;

R⁵ is alkyl, alkenyl, or alkynyl.

In another embodiment, the compound of formula (I) has the following variable assignments:

A=O

R²=R³=R⁴ is H;

R¹ is OC(═O)OR⁵;

R⁵ is alkyl.

In another embodiment, the compound of formula (I) has the following variable assignments:

A=O

R²=R³=R⁴ is H;

R¹ is OC(═O)R⁵;

R⁵ is alkyl, alkyl ether, CCOC(═O)C, alkyl acetate, alkenyl, alkenyl acetate, alkynyl, or alkynyl acetate.

In another embodiment, the compound of formula (I) is set forth according to the following provisions:

A=O

R²=R³=R⁴ is H;

R¹ is C(═O)OR⁵;

R⁵ is alkyl, alkenyl, or alkynyl.

In another embodiment, the compound of formula (I) is set forth according to the following provisions:

A=O

R²=R³=R⁴ is H;

R¹ is OR⁵;

R⁵ is alkyl, alkenyl, or alkynyl.

In another embodiment, the compound of formula (I) is set forth according to the following provisions:

A=O

R¹=R³=R⁴ is H;

R² is OR⁵ or CR⁵;

R⁵ is alkyl, alkenyl, or alkynyl.

In another embodiment, the compound of formula (I) is set forth according to the following provisions:

A=O

R¹=R²=R⁴ is H;

R³ is OC(═O)OR⁵;

R⁵ is alkyl, alkenyl, or alkynyl.

In another embodiment, the compound of formula (I) has the following variable assignments:

A=O

R¹=R²=R⁴ is H;

R³ is OC(═O)OR⁵;

R⁵ is alkyl.

In another embodiment, the compound of formula (I) has the following variable assignments:

A=O

R¹=R²=R⁴ is H;

R³ is OC(═O)R⁵;

R⁵ is alkyl, alkyl ether, CCOC(═O)C, alkyl acetate, alkenyl, alkenyl acetate, alkynyl, or alkynyl acetate.

In another embodiment, the compound of formula (I) is set forth according to the following provisions:

A=O

R¹=R²=R⁴ is H;

R³ is C(═O)OR⁵;

R⁵ is alkyl, alkenyl, or alkynyl.

In another embodiment, the compound of formula (I) is set forth according to the following provisions:

A=O

R¹=R²=R⁴ is H;

R³ is OR⁵;

R⁵ is alkyl, alkenyl, or alkynyl.

In another embodiment, the compound of formula (I) is set forth according to the following provisions:

A=O

R¹=R²=R³ is H;

R⁴ is OC(═O)OR⁵;

R⁵ is alkyl, alkenyl, or alkynyl.

In another embodiment, the compound of formula (I) has the following variable assignments:

A=O

R¹=R²=R³ is H;

R⁴ is OC(═O)OR⁵;

R⁵ is alkyl.

In another embodiment, the compound of formula (I) has the following variable assignments:

A=O

R¹=R²=R³ is H;

R⁴ is OC(═O)R⁵;

R⁵ is alkyl, alkyl ether, CCOC(═O)C, alkyl acetate, alkenyl, alkenyl acetate, alkynyl, or alkynyl acetate.

In another embodiment, the compound of formula (I) is set forth according to the following provisions:

A=O

R¹=R²=R³ is H;

R⁴ is C(═O)OR⁵;

R⁵ is alkyl, alkenyl, or alkynyl.

In another embodiment, the compound of formula (I) is set forth according to the following provisions:

A=O

R¹=R²=R³ is H;

R⁴ is OR⁵;

R⁵ is alkyl, alkenyl, or alkynyl.

In another embodiment, the compound of formula (I) is set forth according to the following provisions:

A=O

R²=R³=R⁴ is H;

R¹ is OCC(═O)N(R⁵)(R⁷) or CC(═O)N(R⁵)(R⁷);

R⁵ and R⁷ are independently selected from alkyl, alkenyl, or alkynyl.

In another embodiment, the compound of formula (I) is set forth according to the following provisions:
A=O

R²═R³=R⁴ is H;

R¹ is OCC(═O)N(R⁵)(R⁷) or CC(═O)N(R⁵)(R⁷);

R⁵ and R⁷ come together to form a C₃-C₁₀ ring which may be substituted by alkyl, alkenyl, or alkynyl.

In another embodiment, the invention provides an insect/pest repellent composition comprising compounds or formulations of the instant disclosure.

In an embodiment, the composition is in a form suitable for topical application to an animal.

The composition of claim 22 that is a cream, gel, spray, liquid or spot-on.

In an embodiment, the invention provides a method for repelling pests comprising the step of applying a compound or composition of any of the disclosed embodiments to animals or a locus.

In another embodiment the animals are birds or mammals.

In another embodiment the mammals are humans, equines, felines, canines, bovines, or caprines.

In another embodiment the animals are equines or bovines.

In another embodiment the animals are humans.

Other obvious variations on the above-recited embodiments will be appreciated by persons skilled in the art.

For convenience, certain terms employed in the Specification, Examples, and appended Claims are collected here.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

As used herein, the word “about”, where it is specifically used to describe a concentration, a mass, a weight, or a volume, is hereby defined to mean “plus or minus 10%” of the stated value.

The following abbreviations shall have the indicated meanings AcOH=Acetic acid; PTSA=p-Toluenesulphonic acid; DAST=Diethylaminosulphur trifluoride DCC=Dicyclohexylcarbodiimide; DCM=Dichloromethane; DEPT=Distortionless Enhancement by Polarisation Transfer; DIEA=Diisopropylethylamine; DMAP=Dimethylaminopyridine; DMF=Dimethylformamide; DMSO=Dimethyl sulphsulphoxide EtOH=Ethanol; Eq=Equivalent; FDPP=Pentafluorophenyl diphenyl phosphinate; HOBt=Hydroxybenzotriazole; HPLC=High Pressure Liquid Chromatography; MeOH=Methanol; MS=Mass spectrometry; MS4A=Molecular sieves 4 Angstrom; MsCl=Mesyl chloride; NBS=N-Bromosuccinimide; NCS=N-Chlorosuccinimide Pyr=Pyridine; Yld=Yield; Rf=Retardation factor; NMR=Nuclear Magnetic Resonance; XR=X-ray; t=Time; A.T.=Ambient temperature; TBAF=Tetrabutylammonium fluoride; TEA=Triethylamine; THF=Tetrahydrofuran; Rt=Retention time; TsCl=Tosyl chloride; TSA=Toluene sulphsulphonic acid; Vol.=Volume; COSY (Correlation Spectroscopy); ¹H-¹H homonuclear scalar coupling; NOESY (Nuclear Overhauser Effect Spectroscopy): ¹H-¹H homonuclear spatial coupling; HMBC (Heteronuclear Multiple Bond Correlation): ¹H-¹³C long-distance correlation; HSQC-TOCSY (Heteronuclear Multiple Quantum Correlation-Total Correlation Spectroscopy.

As used herein, the term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cattle), ovine (e.g., sheep), porcine (e.g., pigs), as well as avians. The term “avian” as used herein refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary, and includes all avians kept as either companion or production animals.

As used herein, the term “aqueous suspension” includes mixtures of insoluble particles in water. Aqueous suspensions may contain the active material in admixture with excipients suitable for the manufacture of aqueous suspensions. Such excipients are suspending agents, for example, colloidal silica, sodium carboxymethylcellulose, methylcellulose, xanthan gum, hydroxy-propylmethylcellulose, sodium alginate, polyinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above.

It will be understood by those of skill in the art that compounds of formula (I) may also be prepared by derivatization of other compounds (I) or by customary modifications of the synthesis routes described.

When the compounds of formula (I) contain suitably acidic or basic residues that enable the formation of veterinarily or pharmaceutically acceptable salts, the compounds may be reacted with suitable acids such as hydrochloric acid, hydrobromic acid, hydroiodic acid, sulfuric acid, phosphoric acid, formic acid, acetic acid, oxalic acid, benzene sulfonic acid, p-toluene sulfonic acid, dodecylbenzene sulfonic acid, methyl bromide, dimethyl sulfate or diethyl sulfate, and the like, typically at a temperature range of about −5° C. to about 150° C., preferably about 0 to about 20° C., in a suitable solvent.

Alternatively, compounds of formula (I) that contain acidic residues may be reacted with suitable bases, including organic amine bases or inorganic bases such as hydroxides, carbonates or bicarbonates of alkali metals or alkaline earth metals.

The formation of the salt is usually conducted in a dissolving or diluting agent. Suitable are e.g. aliphatic hydrocarbons as n-pentane, n-hexane or petrol ether, aromatic hydrocarbons, as toluene or xylenes, or ethers such as diethyl ether, methyl-tert.-butyl ether, tetrahydrofuran or dioxane, further ketones, as acetone, methyl-ethyl-ketone or methyl-isopropyl-ketone, as well as halogenated hydrocarbons as chlorobenzene, methylene chloride, ethylene chloride, chloroform or tetrachloroethylene. Also mixtures of those solvents can be used.

For the preparation of salts of compounds of formula (I) the compounds and salt forming agents are employed usually in a stoichiometric ratio. The excess of one or the other component can be useful.

If individual compounds cannot be prepared via the above-described routes, they can be prepared by derivatization of other compounds or by customary modifications of the synthesis routes described.

The reaction mixtures are typically worked up in a customary manner, for example by mixing a reaction product mixture containing an organic solvent with water, separating the phases, and, if appropriate, purifying the crude products by chromatography, for example on alumina or silica gel. If the intermediates and end products are obtained as solids, they may be purified by recrystallization or digestion.

### Animal Health Applications:

One important aspect of the invention is the use of the compounds of formula (I) or compositions comprising the compounds for the prevention of parasite infestation/infection in or on animals, accomplished via the repulsion of insect or other pest vectors. The compositions of the invention comprise a repellent effective amount of at least one compound of formula (I) in combination with a veterinarily acceptable carrier or diluent and optionally other non-active excipients. The compositions may be in a variety of solid and liquid forms which are suitable for various forms of application or administration to an animal. For example, the veterinary compositions comprising the inventive compounds may be in formulations suitable for oral administration, injectable administration, including subcutaneous and parenteral administration, and topical, pour-on, dermal or subdermal administration. The formulations are intended to be administered to an animal including, but is not limited to, mammals, birds and fish. Examples of mammals include but are not limited to humans, cattle, sheep, goats, llamas, alpacas, pigs, horses, donkeys, dogs, cats and other livestock or domestic mammals. Examples of birds include turkeys, chickens, ostriches and other livestock or domestic birds.

### Veterinary Compositions:

As discussed above, the compositions of the invention may be in a form suitable for oral use (see, e.g., U.S. Pat. No. 4,564,631, which is hereby incorporated by reference in its entirety), dietary supplements, troches, lozenges, chewables, tablets, hard or soft capsules, bolus, emulsions, aqueous or oily suspensions, aqueous or oily solutions, oral formulations, dispersible powders or granules, premixes, syrups or elixirs, enteric formulations or pastes. Compositions intended for oral use may be prepared according to any method known in the art for the manufacture of pharmaceutical compositions and such compositions may contain one or more sweetening agents, bittering agents, flavoring agents, coloring agents and preserving agents in order to provide pharmaceutically elegant and palatable preparations.

Oral products administered to avians are typically, but not exclusively, premix (powder mixed flour) or liquid. A premix product can comprise corn grits or wheat flour, along with appropriate excipients, including preservatives such as antioxidants, and emollients, oils, texturizers, and the like. Inventive formulations may be administered via drinking water, for example, in a chick hatchery setting. Surfactants may be included in such aqueous formulations, both to encourage the inventive compounds to remain in solution prior to being added to the drinking water, and to facilitate even mixing with same.

Tablets may contain the active ingredient in admixture with non-toxic, pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. Nos. 4,256,108; 4,166,452; and 4,265,874 (all incorporated herein by reference in their entirety) to form osmotic therapeutic tablets for controlled release.

Oral formulations include hard gelatin capsules, wherein the active ingredient is mixed with an inert solid diluent, for example, calcium carbonate, calcium phosphate or kaolin. Capsules may also be soft gelatin capsules, wherein the active ingredient is mixed with water or miscible solvents such as propylene glycol, PEGs and ethanol, or an oil medium, for example peanut oil, liquid paraffin, or olive oil.

The compositions of the invention may also be in the form of oil-in-water or water-in-oil emulsions. The oily phase maybe a vegetable oil, for example, olive oil or arachis oil, or a mineral oil, for example, liquid paraffin or mixtures of these. Suitable emulsifying agents include naturally-occurring phosphatides, for example, soy bean, lecithin, and esters or partial esters derived from fatty acids and hexitol anhydrides, for example, sorbitan monoleate, and condensation products of the said partial esters with ethylene oxide, for example, polyoxyethylene sorbitan monooleate. The emulsions may also contain sweetening agents, bittering agents, flavoring agents, and/or preservatives.

In one embodiment, the composition of the invention may be in the form of a microemulsion. Microemulsions are well suited as the liquid carrier vehicle. Microemulsions are quaternary systems comprising an aqueous phase, an oily phase, a surfactant and a cosurfactant. They are translucent and isotropic liquids.

Microemulsions are composed of stable dispersions of microdroplets of the aqueous phase in the oily phase or conversely of microdroplets of the oily phase in the aqueous phase. The size of these microdroplets may be less than 200 nm (1000 to 100,000 nm for emulsions). The interfacial film may be composed of an alternation of surface-active (SA) and co-surface-active (Co-SA) molecules which, by lowering the interfacial tension, allows the microemulsion to be formed spontaneously.

In one embodiment of the oily phase, the oily phase may be formed from mineral or vegetable oils, from unsaturated polyglycosylated glycerides or from triglycerides, or alternatively from mixtures of such compounds. In one embodiment of the oily phase, the oily phase may be comprised of triglycerides; in another embodiment of the oily phase, the triglycerides are medium-chain triglycerides, for example C₈-C₁₀ caprylic/capric triglyceride. In another embodiment of the oily phase may represent a % v/v range of about 2 to about 15%; about 7 to about 10%; and about 8 to about 9% v/v of the microemulsion.

The aqueous phase may include, for example water or glycol derivatives, such as propylene glycol, glycol ethers, polyethylene glycols or glycerol. In one embodiment, the glycol may be propylene glycol, diethylene glycol monoethyl ether, dipropylene glycol monoethyl ether or mixtures thereof. Generally, the aqueous phase will represent a proportion from about 1 to about 4% v/v in the microemulsion.

Surfactants for the microemulsion may include diethylene glycol monoethyl ether, dipropyelene glycol monomethyl ether, polyglycolyzed C₈-C₁₀ glycerides or polyglyceryl-6 dioleate. In addition to these surfactants, the cosurfactants may include short-chain alcohols, such as ethanol and propanol.

Some compounds are common to the three components discussed above, i.e., aqueous phase, surfactant and cosurfactant. However, it is well within the skill level of the practitioner to use different compounds for each component of the same formulation. In one embodiment for the amount of surfactant/cosurfactant, the cosurfactant to surfactant ratio will be from about 1/7 to about 1/2. In another embodiment for the amount of cosurfactant, there will be from about 25 to about 75% v/v of surfactant and from about 10 to about 55% v/v of cosurfactant in the microemulsion.

Oily suspensions may be formulated by suspending the active ingredient in a vegetable oil, for example, arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as sucrose, saccharin or aspartame, bittering agents, and flavoring agents may be added to provide a palatable oral preparation. These compositions may be preserved by the addition of an anti-oxidant such as ascorbic acid, or other known preservatives.

Aqueous suspensions may contain the active material in admixture with excipients suitable for the manufacture of aqueous suspensions. Such excipients include suspending agents, for example, sodium carboxymethylcellulose, methylcellulose, hydroxy-propylmethylcellulose, sodium alginate, polyinylpyrrolidone, gum tragacanth and gum acacia; dispersing or wetting agents include naturally-occurring phosphatide, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents and/or bittering agents, such as those set forth above.

Dispersible powders and granules suitable for preparation of an aqueous suspension by the addition of water may provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring and coloring agents, may also be present.

Syrups and elixirs may be formulated with sweetening agents, for example, glycerol, propylene glycol, sorbitol or sucrose. Such formulations may also contain a demulcent, a preservative, flavoring agent(s) and/or coloring agent(s).

In another embodiment of the invention, the composition may be in paste form. Examples of embodiments in a paste form include, but are not limited to, those described in U.S. Pat. Nos. 6,787,342 and 7,001,889 (each of which are incorporated herein by reference). In addition to the compounds of the invention, the paste may further contain fumed silica; a viscosity modifier; a carrier; optionally, an absorbent; and optionally, a colorant, stabilizer, surfactant, or preservative.

In one embodiment of the formulation, the formulation may be a paste containing the compounds of the invention, fumed silica, a viscosity modifier, an absorbent, a colorant; and a hydrophilic carrier which is triacetin, a monoglyceride, a diglyceride, or a triglyceride.

The paste may also include a viscosity modifier. Suitable viscosity modifiers include, but are not limited to, polyethylene glycols (PEG) including, but not limited to, PEG 200, PEG 300, PEG 400, PEG 600; monoethanolamine, triethanolamine, glycerol, propylene glycol, polyoxyethylene (20) sorbitan mono-oleate (polysorbate 80 or Tween 80), or polyoxamers (e.g., Pluronic L 81); an absorbent such as magnesium carbonate, calcium carbonate, starch, and cellulose and its derivatives; and a colorant including, but not limited to, titanium dioxide iron oxide, or FD&C Blue #1 Aluminum Lake.

In some embodiments, the compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example, as a solution in 1,3-butane diol. Among the acceptable vehicles and solvents that may be employed are water, Ringer's solution and isotonic sodium chloride solution. Cosolvents such as ethanol, propylene glycol, glycerol formal or polyethylene glycols may also be used. Preservatives, such as phenol or benzyl alcohol, may be used.

In addition, sterile, fixed oils may be conventionally employed as a solvent or suspending medium. For this purpose any bland fixed oil may be employed including synthetic mono- or diglycerides. In addition, fatty acids such as oleic acid find use in the preparation of injectables.

Topical, dermal and subdermal formulations may include, by way of non-limiting example, emulsions, creams, ointments, gels, pastes, powders, shampoos, pour-on formulations, ready-to-use formulations, spot-on solutions and suspensions, dips and sprays. Topical application of an inventive compound or of a composition including at least one inventive compound among active agent(s) therein, in the form of a spot-on, spray-on or pour-on composition, may allow for the inventive composition to be absorbed through the skin to achieve systemic levels, distributed through the sebaceous glands or on the surface of the skin achieving levels throughout the coat. When the compound is distributed through the sebaceous glands, they may act as a reservoir, whereby there may be a long-lasting effect (up to several months) effect. Spot-on formulations are typically applied in a localized region which refers to an area other than the entire animal. In one embodiment, the location may be between the shoulders. In another embodiment it may be a stripe, e.g. a stripe from head to tail of the animal.

Pour-on formulations are described in U.S. Pat. No. 6,010,710, also incorporated herein by reference. Pour-on formulations may be advantageously oily, and generally comprise a diluent or vehicle and also a solvent (e.g. an organic solvent) for the active ingredient if the latter is not soluble in the diluent.

Organic solvents that can be used in the invention include, but are not limited to, acetyltributyl citrate, fatty acid esters such as the dimethyl ester, diisobutyl adipate, acetone, acetonitrile, benzyl alcohol, ethyl alcohol, butyl diglycol, dimethylacetamide, dimethylformamide, dimethyl sulfoxide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol, triacetin, C₁-C₁₀ esters of carboxylic acids such as butyl or octyl acetate, and diethyl phthalate, or a mixture of at least two of these solvents.

The solvent will be used in proportion with the concentration of the active agent compound and its solubility in this solvent. It will be sought to have the lowest possible volume. The vehicle makes up the difference to 100%.

A vehicle or diluent for the formulations may include dimethyl sulfoxide (DMSO), glycol derivatives such as, for example, propylene glycol, glycol ethers, polyethylene glycols or glycerol. As vehicle or diluent, mention may also be made of plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈ to C₁₂) triglycerides.

In another embodiment of the invention, an emollient and/or spreading and/or film-forming agent may be added. In one embodiment, the emollient and/or spreading and/or film-forming agent may be:

(a) polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose, silicone oils, polydiorganosiloxane oils (such as polydimethylsiloxane (PDMS) oils), for example those containing silanol functionalities, or a 45V2 oil,

(b) anionic surfactants such as alkaline stearates, sodium, potassium or ammonium stearates; calcium stearate, triethanolamine stearate; sodium abietate; alkyl sulphates (e.g. sodium lauryl sulphate and sodium cetyl sulphate); sodium dodecylbenzenesulphonate, sodium dioctylsulphosuccinate; fatty acids (e.g. those derived from coconut oil),

(c) cationic surfactants include water-soluble quaternary ammonium salts of formula N⁺R′R″R′″R″″, Y⁻ in which the radicals R are optionally hydroxylated hydrocarbon radicals and Y⁻ is an anion of a strong acid such as the halide, sulphate and sulphonate anions; cetyltrimethylammonium bromide is among the cationic surfactants which can be used,

(d) amine salts of formula N⁺HR′R″R′″ in which the radicals R are optionally hydroxylated hydrocarbon radicals; octadecylamine hydrochloride is among the cationic surfactants which can be used,

(e) nonionic surfactants such as sorbitan esters, which are optionally polyoxyethylenated (e.g. polysorbate 80), polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide,

(f) amphoteric surfactants such as the substituted lauryl compounds of betaine; or

(g) a mixture of at least two of these agents.

In one embodiment of the amount of emollient, the emollient used may be in a proportion of from about 0.1 to 50% or 0.25 to 5%, by volume. In another embodiment, the emollient used may be in a proportion of from about 0.1% to about 30%, about 1% to about 30%, about 1% to about 20%, or about 5% to about 20% by volume.

In another embodiment of the invention, the composition may be in ready-to-use solution form as is described in U.S. Pat. No. 6,395,765, incorporated herein by reference. In addition to the compounds of the invention, the ready-to-use solution may contain a crystallization inhibitor and an organic solvent or a mixture of organic solvents. In some embodiments, water may be included with the organic solvent.

In various embodiments of the invention, the compositions may include a crystallization inhibitor in an amount of about 1 to about 50% (w/v) or about 5 to about 40% (w/v) based on the total weight of the formulation. In other embodiments, the amount of crystallization inhibitor in the inventive formulations may be about 1% to about 30%, about 5% to about 20%, about 1% to about 15%, or about 1% to about 10% (w/w). The type of crystallization inhibitor used in the inventive formulations is not limited as long as it functions to inhibit crystallization of the active or inactive agents from the formulation. For example, in certain embodiments of the invention, a solvent or co-solvent of the formulation may also function as a crystallization inhibitor if it sufficiently inhibits the formation of crystals from forming over time when the formulation is administered.

Crystallization inhibitors which are useful for the invention include, but are not limited to:

(a) polyvinylpyrrolidone, polyvinyl alcohols, copolymers of vinyl acetate and vinylpyrrolidone, polyethylene glycols, benzyl alcohol, dimethylformamide, dimethylacetamide, dimethylsulfoxide, 2-pyrrolidone, N-methylpyrrolidone, mannitol, glycerol, sorbitol or polyoxyethylenated esters of sorbitan; lecithin or sodium carboxymethylcellulose; or acrylic derivatives, such as acrylates or methacrylates or polymers or copolymers thereof, polyethyleneglycols (PEG) or polymers containing polyethyleneglycols, such as glycofurol and the like, and others;

(b) anionic surfactants, such as alkaline stearates (e.g. sodium, potassium or ammonium stearate); calcium stearate or triethanolamine stearate; sodium abietate; alkyl sulphates, which include but are not limited to sodium lauryl sulphate and sodium cetyl sulphate; sodium dodecylbenzenesulphonate or sodium dioctyl sulphosuccinate; or fatty acids (e.g. coconut oil);

(c) cationic surfactants, such as water-soluble quaternary ammonium salts of formula N⁺R′R″R′″R″″Y, in which the R radicals are identical or different optionally hydroxylated hydrocarbon radicals and Y⁻ is an anion of a strong acid, such as halide, sulphate and sulphonate anions; cetyltrimethylammonium bromide is one of the cationic surfactants which can be used;

(d) amine salts of formula N⁺HR′R″R′″, in which the R radicals are identical or different optionally hydroxylated hydrocarbon radicals; octadecylamine hydrochloride is one of the cationic surfactants which can be used;

(e) non-ionic surfactants, such as optionally polyoxyethylenated esters of sorbitan, e.g. Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide;

(f) amphoteric surfactants, such as substituted lauryl compounds of betaine;

(g) a mixture of at least two of the compounds listed in (a)-(f) above; or

(h) an organic solvent or mixture of solvents which inhibit the formation of crystals or amorphous solid after the formulation is administered.

In one embodiment of the crystallization inhibitor, a crystallization inhibitor pair will be used. Such pairs include, for example, the combination of a film-forming agent of polymeric type and of a surface-active agent. These agents will be selected from the compounds mentioned above as crystallization inhibitor.

In some embodiments, the organic solvent(s) may have a dielectric constant of between about 10 and about 35 or between about 20 and about 30. In other embodiments, the organic solvent may have a dielectric constant of between about 10 and about 40 or between about 20 and about 30. The content of this organic solvent or mixture of solvents in the overall composition is not limited and will be present in an amount sufficient to dissolve the desired components to a desired concentration. As discussed above, the organic solvent may also function as a crystallization inhibitor in the formulation.

In some embodiments, one or more of the organic solvent(s) may have a boiling point of below about 100° C., or below about 80° C. In other embodiments, the organic solvent(s) may have a boiling point of below about 300° C., below about 250° C., below about 230° C., below about 210° C. or below about 200° C.

In some embodiments where there is a mixture of solvents, i.e. a solvent and a co-solvent, the solvents may be present in the composition in a weight/weight (W/W) ratio of about 1/50 to about 1/1. Typically the solvents will be in a ratio of about 1/30 to about 1/1, about 1/20 to about 1/1, or about 1/15 to about 1/1 by weight. Preferably, the two solvents will be present in a weight/weight ratio of about 1/15 to about 1/2. In some embodiments, at least one of the solvents present may act as to improve solubility of the active agent or as a drying promoter. In particular embodiments, at least one of the solvents will be miscible with water.

The formulation may also comprise an antioxidizing agent intended to inhibit oxidation in air, this agent may be present in a proportion of about 0.005 to about 1% (w/v), about 0.01 to about 0.1%, or about 0.01 to about 0.05%.

In one embodiment of the film-forming agent, the agents are of the polymeric type which include but are not limited to the various grades of polyvinylpyrrolidone, polyvinyl alcohols, and copolymers of vinyl acetate and of vinylpyrrolidone.

In one embodiment of the surface-active agents, the agents include but are not limited to those made of non-ionic surfactants; in another embodiment of the surface active agents, the agent is a polyoxyethylenated esters of sorbitan and in yet another embodiment of the surface-active agent, the agents include the various grades of polysorbate, for example Polysorbate 80.

In another embodiment of the invention, the film-forming agent and the surface-active agent may be incorporated in similar or identical amounts within the limit of the total amounts of crystallization inhibitor mentioned elsewhere.

The crystallization inhibitor inhibits the formation of crystals on the coat, and improves the maintenance of the cosmetic appearance of the skin or fur; that is to say without a tendency towards sticking or towards a sticky appearance, despite the high concentration of active material. Substances other than those mentioned herein may be used as crystallization inhibitors in the present invention. In one embodiment, the effectiveness of the crystallization inhibitor may be demonstrated by a test according to which 0.3 mL of a solution comprising 10% (w/v) of the active agent in an appropriate solvent as defined above, and 10% (w/v) of the compound acting as a crystallization inhibitor are placed on a glass slide at 20° C. for 24 hours, after which fewer than 10 crystals, preferably 0 crystals, are seen with the naked eye on the glass slide.

In one embodiment of the antioxidizing agents, the agents are those conventional in the art and include but are not limited to butylated hydroxyanisole, butylated hydroxytoluene, ascorbic acid, sodium metabisulphite, propyl gallate, sodium thiosulphate or a mixture of at least two compounds with antioxidant properties.

The formulation adjuvants discussed above are well known to the practitioner in this art and may be obtained commercially or through known techniques. These concentrated compositions are generally prepared by simple mixing of the constituents as defined above; advantageously, the starting point is to mix the active material in the main solvent and then the other ingredients or adjuvants are added.

The volume of the formulation applied will depend on the type of animal and the size of the animal as well as the strength of the formulation and the potency of the active agents. In one embodiment, an amount of about 0.1 to about 20 ml of the formulation may be applied to the animal. In other embodiment for the volume, the volume may be about 0.1 to about 10 ml, about 0.1 to about 5 ml, about 0.5 ml to about 10 ml, or about 0.3 to about 3 ml.

In another embodiment of the invention, application of a spot-on formulation according to the present invention may also provide long-lasting and broad-spectrum efficacy when the solution is applied to the mammal or bird. The spot-on formulations provide for topical administration of a concentrated solution, suspension, microemulsion or emulsion for intermittent application to a spot on the animal, generally between the two shoulders (solution of spot-on type).

For spot-on formulations, the carrier may be a liquid carrier vehicle as described in U.S. Pat. No. 6,426,333 (incorporated herein by reference), which in one embodiment of the spot-on formulation may comprise a solvent or mixture of solvents including, but not limited to, acetone, an aliphatic alcohol such as methanol, ethanol, propanol, butanol, isopropanol, pentanol, hexanol, heptanol, octanol, nonanol, cyclopentanol, cyclohexanol, ethylene glycol, propylene glycol and the like; an aromatic alcohol such as phenol, cresol, naphthol, benzyl alcohol and the like; acetonitrile, butyl diglycol, an organic amide such as dimethylacetamide, dimethylformamide, monomethylacetamide, 2-pyrrolidone, N-methylpyrrolidone, vinylpyrrolidone and the like; dimethylsulfoxide (DMSO), a glycol polymer or an ether thereof, such as polyethylene glycol (PEG) of various grades, polypropylene glycols of various grades, dipropylene glycol n-butyl ether, ethylene glycol monoethyl ether, ethylene glycol monomethyl ether, dipropylene glycol monomethyl ether, diethylene glycol monoethyl ether, ethylene glycol, diethyl phthalate fatty acid esters, such as the diethyl ester or diisobutyl adipate, or a mixture of at least two of these solvents.

The liquid carrier vehicle may optionally contain a crystallization inhibitor including, but not limited to, those described in (a) to (h) above, or a compound that may act both as a solvent and a crystallization inhibitor (as defined above), or a mixture of these crystallization inhibitors.

Spot-on formulations may be prepared by dissolving the active ingredients into the pharmaceutically or veterinary acceptable vehicle. Alternatively, the spot-on formulation may be prepared by encapsulation of the active ingredient to leave a residue of the therapeutic agent on the surface of the animal. These formulations will vary with regard to the weight of the therapeutic agent in the combination depending on the species of host animal to be treated, the severity and type of infection and the body weight of the host.

Dosage forms may typically contain from about 0.1 mg to about 5 g. In other embodiments, the dosage form may contain about 0.5 mg to about 5 g of an active agent. In one embodiment of the dosage form, the dosage may contain from about 1 mg to about 500 mg of an active agent, typically about 25 mg, about 50 mg, about 100 mg, about 200 mg, about 300 mg, about 400 mg, about 500 mg, about 600 mg, about 800 mg, or about 1000 mg.

In one embodiment of the invention, the active agent may be present in the formulation at a concentration of about 0.05 to about 10% weight/volume. In another embodiment of the invention, the active agent may be present in the formulation as a concentration from about 0.1 to about 2% weight/volume. In yet another embodiment of the invention, the active agent may be present in the formulation as a concentration from about 0.25 to about 1.5% weight/volume. In still another embodiment of the invention, the active agent may be present in the formulation as a concentration about 1% weight/volume.

### Methods of Treatment:

As discussed above, the compounds of formula (I) or (Ia) are effective in repelling insects and pests, and therefore may prevent insect/pest-borne infestations in animals or humans. In one embodiment, the invention provides a method for repelling insects or other pests away from an animal, comprising administering a repellent effective amount of a compound of formula (I) or (Ia), or veterinarily or pharmaceutically acceptable salts thereof, or a composition comprising the compounds, to the animal.

In still another embodiment of the invention, a method is provided for repulsion of insects/pests at a locus, which comprises administering or applying a repellent effective amount of a compound of formula (I) or (Ia), or veterinarily acceptable salts thereof, to the locus. With respect to animal health applications, “locus” is intended to mean a habitat, breeding ground, area, material or environment in which a parasite is growing or may grow, including in or on an animal.

Mammals which can be treated include but are not limited to humans, cats, dogs, cattle, chickens, cows, deer, goats, horses, llamas, pigs, sheep and yaks. In one embodiment of the invention, the mammals treated are humans, cats or dogs.

When an anthelmintic agent is added to the composition of the invention, the composition can also be used to treat against endoparasites such as those helminths selected from the group consisting of Anaplocephala, Ancylostoma, Anecator, Ascaris, Capillaria, Cooperia, Dipylidium, Dirofilaria, Echinococcus, Enterobius, Fasciola, Haemonchus, Oesophagostumum, Ostertagia, Oxyuris spp., Toxocara, Strongyloides, Strongylus spp., Toxascaris, Trichinella, Trichuris, and Trichostrongylus. The inventive compounds are particularly effective against organisms from the class of Protozoa, for example, Eimeria spp. and Plasmodia spp.

In each aspect of the invention, the compounds and compositions of the invention can be applied against a single organism/parasite or combinations thereof.

Additional pharmaceutical, pesticidal or veterinarily active ingredients, which include, but are not limited to, parasiticidals including acaricides, anthelmintics, endectocides and insecticides, may also be added to the compositions of the invention. Anti-parasitic agents may include both ectoparasiticidal and endoparasiticidal agents. Veterinary pharmaceutical agents are well-known in the art (see e.g. Plumb' Veterinary Drug Handbook, 5^{th} Edition, ed. Donald C. Plumb, Blackwell Publishing, (2005) or The Merck Veterinary Manual, 9^{th} Edition, (January 2005)) and include but are not limited to acarbose, acepromazine maleate, acetaminophen, acetazolamide, acetazolamide sodium, acetic acid, acetohydroxamic acid, acetylcysteine, acitretin, acyclovir, afoxonil, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitraz, amitriptyline, amlodipine besylate, ammonium chloride, ammonium molybdenate, amoxicillin, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide+/−clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, dichlorvos, diclofenac sodium, dicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fenbendazole, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inaminone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morantel tartrate, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprozole, ondansetron, opiate antidiarrheals, orbifloxacin, oxacillin sodium, oxazepam, oxfendazole, oxibutynin chloride, oxymorphone, oxytretracycline, oxytocin, pamidronate disodium, pancreplipase, pancuronium bromide, paromomycin sulfate, parozetine, pencillamine, penicillins including penicillin G and penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenyloin sodium, pheromones, parenteral phosphate, phytonadione/vitamin K-1, pimobendan, piperazine, pirlimycin, piroxicam, polysulfated glycosaminoglycan, ponazuril, potassium chloride, pralidoxime chloride, praziquantel, prazosin, prednisolone/prednisone, primidone, procainamide, procarbazine, prochlorperazine, propantheline bromide, propionibacterium acnes injection, propofol, propranolol, protamine sulfate, pseudoephedrine, psyllium hydrophilic mucilloid, pyrantel pamoate, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/1-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiabendazole, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocamide, tolazoline, telfenamic acid, topiramate, tramadol, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof.

In one embodiment of the invention, arylpyrazole compounds may be added to the compositions of the invention. Arylpyrazoles may include but are not limited to those described in U.S. Pat. Nos. 6,001,384; 6,010,710; 6,083,519; 6,096,329; 6,174,540; 6,685,954 and 6,998,131, all of which are hereby incorporated by reference in their entirety, —each assigned to Merial, Ltd., Duluth, Ga.). A particularly preferred arylpyrazole compound that may be combined with the compounds of the invention is fipronil (5-amino-1-[2,6-dichloro-4-(trifluoromethyl)phenyl]-4-(trifluoromethylsulfinyl)pyrazole-3-carbonitrile, CAS No. 120068-37-3).

In another embodiment of the invention, nodulisporic acid and its derivatives (a class of known acaricidal, anthelmintic, anti-parasitic and insecticidal agents) may be added to the compositions of the invention. These compounds are used to treat or prevent infections in humans and animals and are described, for example, in U.S. Pat. Nos. 5,399,582, 5,962,499, 6,221,894 and 6,399,786, all of which are hereby incorporated by reference in their entirety. The compositions may include one or more of the known nodulisporic acid derivatives in the art, including all stereoisomers, such as those described in the literature cited above.

In another embodiment, anthelmintic compounds of the amino acetonitrile class (AAD) of compounds such as monepantel (ZOLVIX) and the like may be added to the compositions of the invention. These compounds are described, for example, in WO 2004/024704; Sager et al., Veterinary Parasitology, 2009, 159, 49-54; Kaminsky et al., Nature vol. 452, 13 Mar. 2008, 176-181.

The compositions of the invention may also be combined with paraherquamide compounds and derivatives of these compounds, including derquantel (see Ostlind et al., Research in Veterinary Science, 1990, 48, 260-61; and Ostlind et al., Medical and Veterinary Entomology, 1997, 11, 407-408). The paraherquamide family of compounds are known class of compounds that include a spirodioxepino indole core with activity against certain parasites (see Tet. Lett. 1981, 22, 135; J. Antibiotics 1990, 43, 1380, and J. Antibiotics 1991, 44, 492). In addition, the structurally related marcfortine family of compounds, such as marcfortines A-C, are also known and may be combined with the formulations of the invention (see J. Chem. Soc.-Chem. Comm. 1980, 601 and Tet. Lett. 1981, 22, 1977). Further references to the paraherquamide derivatives can be found, for example, in WO 91/09961, WO 92/22555, WO 97/03988, WO 01/076370, WO 09/004,432, U.S. Pat. No. 5,703,078 and U.S. Pat. No. 5,750,695, all of which are hereby incorporated by reference in their entirety.

In another embodiment, the compositions of the invention may be combined with cyclo-depsipeptide anthelmintic compounds including emodepside (see Willson et al., Parasitology, January 2003, 126(Pt 1):79-86).

In another embodiment of the invention, the class of acaricides or insecticides known as insect growth regulators (IGRs) may also be added to the compositions of the invention. Compounds belonging to this group are well known to the practitioner and represent a wide range of different chemical classes. These compounds all act by interfering with the development or growth of the insect pests. Insect growth regulators are described, for example, in U.S. Pat. No. 3,748,356; U.S. Pat. No. 3,818,047; U.S. Pat. No. 4,225,598; U.S. Pat. No. 4,798,837; U.S. Pat. No. 4,751,225, EP 0 179 022 or GB 2 140 010 as well as U.S. Pat. Nos. 6,096,329 and 6,685,954, all of which are hereby incorporated by reference in their entirety. Examples of IGRs suitable for use may include but are not limited to methoprene, pyriproxyfen, hydroprene, cyromazine, fluazuron, lufenuron, novaluron, pyrethroids, formamidines and 1-(2,6-difluorobenzoyl)-3-(2-fluoro-4-(trifluoromethyl)phenylurea.

An anthelmintic agent that may be combined with the compositions of the invention may be a benzenedisulfonamide compound, which includes but is not limited to clorsulon; or a cestodal agent, which includes but is not limited to praziquantel, pyrantel or morantel.

In some embodiments, a parasiticidal agent that may be combined with the compositions of the invention may be a biologically active peptide or protein including, but not limited to, depsipeptides, which act at the neuromuscular junction by stimulating presynaptic receptors belonging to the secretin receptor family resulting in the paralysis and death of parasites. In one embodiment of the depsipeptide, the depsipeptide may be emodepside.

In other embodiments, an insecticidal agent that may be combined with the compositions of the invention may be a spinosyn (e.g. spinosad) or a substituted pyridylmethyl derivative compound such as imidacloprid. Agents of this class are described above, and for example, in U.S. Pat. No. 4,742,060 or in EP 0 892 060, both of which are hereby incorporated by reference in their entirety.

For endoparasites, parasiticides which may be combined include but are not limited to pyrantel, morantel, the benzimidazoles (including albendazole, cambendazole, thiabendazole, fenbendazole, febantel, oxfendazole, oxibendazole, triclabendazole, mebendazole and netobimin), levamisole, closantel, rafoxanide, nitroxynil, disophenol and paraherquamide. For ectoparasites, insecticides which may be combined also include but are not limited to pyrethroids, organophosphates and neonicotinoids such as imidacloprid, as well as compounds such as metaflumizone, amitraz and ryanodine receptor antagonists.

The compositions of the invention may also comprise an antiparasitic macrocyclic lactone compound in combination with the active compound of the invention. The macrocyclic lactones include, but are not limited to, avermectins, such as abamectin, dimadectin, doramectin, emamectin, eprinomectin, ivermectin, latidectin, lepimectin, selamectin, ML-1,694,554 and milbemycins, such as milbemectin, milbemycin D, moxidectin and nemadectin. Also included are the 5-oxo and 5-oxime derivatives of said avermectins and milbemycins. Examples of compositions comprising macrocyclic lactones include but are not limited to those described in U.S. Pat. Nos. 6,426,333; 6,482,425; 6,962,713 and 6,998,131, all of which are incorporated by reference in their entirety; —each assigned to Merial, Ltd., Duluth, Ga.

The macrocyclic lactone compounds are known in the art and can easily be obtained commercially or through synthesis techniques known in the art. Reference is made to the widely available technical and commercial literature. For avermectins, ivermectin and abamectin, reference may be made, for example, to the work “Ivermectin and Abamectin”, 1989, by M. H. Fischer and H. Mrozik, William C. Campbell, published by Springer Verlag, or Albers-Schonberg et al. (1981), “Avermectins Structure Determination”, J. Am. Chem. Soc., 103, 4216-4221. For doramectin, “Veterinary Parasitology”, vol. 49, No. 1, July 1993, 5-15 may be consulted. For milbemycins, reference may be made, inter alia, to Davies H. G. et al., 1986, “Avermectins and Milbemycins”, Nat. Prod. Rep., 3, 87-121, Mrozik H. et al., 1983, Synthesis of Milbemycins from Avermectins, Tetrahedron Lett., 24, 5333-5336, U.S. Pat. No. 4,134,973 and EP 0 677 054.

Macrocyclic lactones are either natural products or are semi-synthetic derivatives thereof. The structure of the avermectins and milbemycins are closely related, e.g., by sharing a complex 16-membered macrocyclic lactone ring. The natural product avermectins are disclosed in U.S. Pat. No. 4,310,519 and the 22,23-dihydro avermectin compounds are disclosed in U.S. Pat. No. 4,199,569, each of which is incorporated herein by reference. Mention is also made of U.S. Pat. Nos. 4,468,390, 5,824,653, EP 0 007 812 A1, U.K. Patent Specification 1 390 336, EP 0 002 916, and New Zealand Patent No. 237 086, inter alia, all of which are incorporated by reference in their entirety. Naturally occurring milbemycins are described in U.S. Pat. No. 3,950,360 as well as in the various references cited in “The Merck Index” 12^{th} ed., S. Budavari, Ed., Merck & Co., Inc. Whitehouse Station, N.J. (1996). Latidectin is described in the “International Nonproprietary Names for Pharmaceutical Substances (INN)”, WHO Drug Information, vol. 17, no. 4, pp. 263-286, (2003). Semisynthetic derivatives of these classes of compounds are well known in the art and are described, for example, in U.S. Pat. Nos. 5,077,308, 4,859,657, 4,963,582, 4,855,317, 4,871,719, 4,874,749, 4,427,663, 4,310,519, 4,199,569, 5,055,596, 4,973,711, 4,978,677, 4,920,148 and EP 0 667 054, all of which are incorporated by reference in their entirety.

In yet another embodiment of the invention, adulticide insecticides and acaricides can also be added to the composition of the invention. These include pyrethrins (which include cinerin I, cinerin II, jasmolin I, jasmolin II, pyrethrin I, pyrethrin II and mixtures thereof) and pyrethroids, organophosphate (which included but are not limited to chlorfenvinphos, crotoxyphos, dichlorvos, heptenophos, mevinphos, monocrotophos, naled, TEPP, tetrachlorvinphos) and carbamates (which include but are not limited to benomyl, carbanolate, carbaryl, carbofuran, meththiocarb, metolcarb, promacyl, propoxur, aldicarb, butocarboxim, oxamyl, thiocarboxime and thiofanox).

In addition to the other active agents mentioned above, combinations of two or more active agents may be used with the compounds of the invention in a composition to treat a desired spectrum of pests and parasites. It would be well within the skill level of the practitioner to decide which individual compound can be used in the inventive formulation to treat a particular infection of an insect.

### REFERENCES

Anna Petherick (2008-03-13). “How DEET jams insects' smell sensors”. Nature News. http://www.nature.com/news/2008/080313/full/news.2008.672.html. Retrieved 2008-03-16.
Mathias Ditzen, Maurizio Pellegrino, Leslie B. Vosshall (2008). “Insect Odorant Receptors Are Molecular Targets of the Insect Repellent DEET”. Sciencexpress 319 (5871): 1838. doi:10.1126/science.1153121. PMID 18339904.
Syed, Z.; Leal, W S (2008). “Mosquitoes smell and avoid the insect repellent DEET”. Proc. Natl. Acad. Sci. USA 105 (36): 13598. doi:10.1073/pnas.0805312105.
Matsuda, Brent M.; Surgeoner, Gordon A.; Heal, James D.; Tucker, Arthur O.; Maciarello, Michael J. (1996). “Essential oil analysis and field evaluation of the citrosa plant “Pelargonium citrosum” as a repellent against populations of Aedes mosquitoes”. Journal of the American Mosquito Control Association 12 (1): 69-74. PMID 8723261.
University of North Carolina (3 Jul. 2002). “Independent study: DEET products superior for fending off mosquito bites”. Press release. http://www.unc.edu/news/archives/jul02/fradin070202.htm.
“Protection against Mosquitoes, Ticks, Fleas and Other Insects and Arthropods”. Travelers'Health-Yellow Book. Centers for Disease Control and Prevention. 2009-02-05. http://wwwn.cdc.gov/travel/yellowBookCh2-InsectsArthropods.aspx.
“Insect Repellent Use and Safety”. West Nile Virus. Centers for Disease Control and Prevention. 2007-01-12. http://www.cdc.gov/ncidod/dvbid/westnile/qa/insect repellent.htm.
CDC. “Insect Repellent Use and Safety”. West Nile Virus. Centers for Disease Control and Prevention. 2007-01-12. http://www.cdc.gov/ncidod/dvbid/westnile/qa/insect_repellent.htm.
“Reregistration Eligibility Decision: DEET.” U.S. Environmental Protection Agency, Office of Prevention, Pesticides, and Toxic Substances. September 1998. pp 39-40
“DEET”. Pesticide Information Profile. EXTOXNET. October 1997. http://pmep.cce.cornell.edu/profiles/extoxnet/carbaryl-dicrotophos/deet-ext.html. Retrieved 2007-09-26.
“Insect Repellents”. Healthy Living. Health Canada. August 2009. http://www.hc-sc.gc.ca/hl-vs/iyh-vsv/life-vie/insect-eng.php. Retrieved 2010-07-09.
“Re-evaluation Decision Document: Personal insect repellents containing DEET (N,N-diethyl-m-toluamide and related compounds)”. Consumer Product Safety. Health Canada. 2002-04-15. http://www.hc-sc.gc.ca/cps-spc/pubs/pest/_decisions/rrd2002-01/index-eng.php. Retrieved 2010-07-09.
Corbel et al.; Stankiewicz, M; Pennetier, C; Fournier, D; Stojan, J; Girard, E; Dimitrov, M; Molgó, J et al. (2009). “Evidence for inhibition of cholinesterases in insect and mammalian nervous systems by the insect repellent deet”. BMC Biology 7:47 7: 47. doi:10.1186/1741-7007-7-47. PMC 2739159. PMID 19656357. http://www.biomedcentral.com/1741-7007/7/47.
Purves, Dale, George J. Augustine, David Fitzpatrick, William C. Hall, Anthony-Samuel LaMantia, James O. McNamara, and Leonard E. White (2008). Neuroscience. 4th ed. Sinauer Associates. pp. 121-2. ISBN 978-0-87893-697-7.
Moss (1996). “Synergism of Toxicity of N,N-Diethyl-m-toluamide to German Cockroaches (Othoptera: Blattellidae) by Hydrolytic Enzyme Inhibitors.”. J. Econ. Entomol. 89 (5): 1151-1155. PMID 17450648.
U.S. Environmental Protection Agency. 1980. Office of Pesticides and Toxic Substances. N,N-diethyl-m-toluamide (Deet) Pesticide Registration Standard. December, 1980.83 pp.
Mathai, A T; Pillai, K S; Deshmukh, P B (1989). “Acute toxicity of deet to a freshwater fish, Tilapia mossambica: Effect on tissue glutathione levels”. Journal of Environmental Biology 10 (2): 87-91. http://md1.csa.com/partners/viewrecord.php?requester=gs&collection=ENV&recid=2427 671&q=DEET+fish+-permethrin&uid=791055180&setcookie=yes.
J. Seo, Y. G. Lee, S. D. Kim, C. J. Cha, J. H. Ahn and H. G. Hur (2005). “Biodegradation of the Insecticide N,N-Diethyl-m-Toluamide by Fungi: Identification and Toxicity of Metabolites”. Archives of Environmental Contamination and Toxicology 48 (3): 323-328. doi:10.1007/s00244-004-0029-9. PMID 15750774.
“Errol Zeiger, Raymond Tice, Brigette Brevard, (1999) N,N-Diethyl-m-toluamide (DEET) [134-62-3]—Review of Toxicological Literature” (PDF). http://ntp.niehs.nih.gov/ntp/htdocs/Chem_Background/ExSumPdf/DEET.pdf. Retrieved July 20.

The invention will now be further described by way of the following non-limiting examples.

### EXAMPLES

The following examples are provided to illustrate certain embodiments of the invention and are not to be construed in any way as limiting the scope of the invention.

### Example 1

### Coumarin Ether Derivatives

TABLE 2
Coumarin ethers tested in vivo

| Reaction | | Iso- | Mol. | | |
|---|---|---|---|---|---|
| | | lated | | | |
| number | Ether | yield | Mass | LogP | In vivo tests |
| EV04036 | 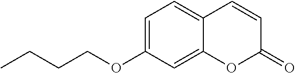 | 82% | 218.09 | 2.95 | Tube EtOH: 50% Tube product: 50% |
| EV04032 | 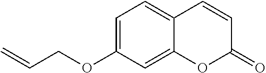 | 35% | 202.06 | 2.36 | Tube EtOH: 59.28% Tube product: 40.72% Tube AcOEt: 62.25% Tube product: 37.75% |
| EV04054 | 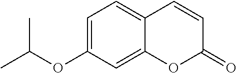 | 83% | 204.08 | 2.40 | Tube EtOH: 82.74% Tube product: 17.26% |
| EV04058 | 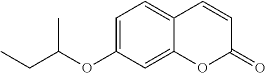 | 91% | 218.09 | 2.92 | 20%: Tube EtOH: 88.02% Tube product: 11.98% 10%: Tube EtOH: 91.22%: Tube product: 8.78% |
| EV04070 | 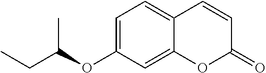 | 94% | 218.09 | 2.92 | 20%: Tube EtOH: 91.34% Tube product: 8.66% 10%: Tube EtOH: 92.79% Tube product: 7.21% |
| EV04084 | 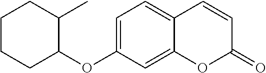 | 35% | 258.12 | 3.79 | Tube EtOH: 49.48% Tube product: 50.52% |
| EV04090 | 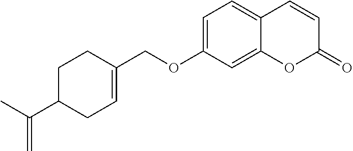 | 73% | 296.14 | 4.08 | Tube CH₂Cl₂: 70.20% Tube product: 29.80% |
| EV04094 | 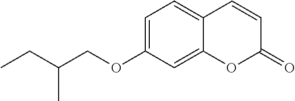 | 85% | 232.11 | 3.31 | 20%: Tube EtOH: 86.47% Tube product: 13.53% 10%: Tube EtOH: 89.63% Tube product: 10.37% |
| EV04114 | 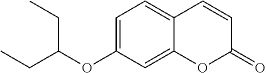 | 99% | 232.11 | 3.44 | 20%: Tube EtOH: 93.09% Tube product: 6.91% 10%: Tube EtOH: 88.00% Tube product: 12.00% |
| EV04188 | 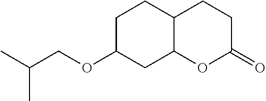 | 28% | 226.16 | 2.31 | Tube EtOH: 82.35% Tube product: 17.65% |
| EV05184 | 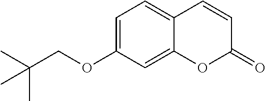 | — | 232.11 | 3.25 | — |
| EV06018 | 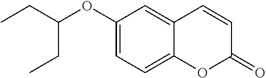 | 79% | 232.11 | 3.44 | Tube EtOH: 71.50% Tube product: 28.50% Tube AcOEt: 81.07% Tube product: 18.93% |
| EV06026 | 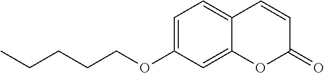 | 100% | 232.11 | 3.39 | 20%: Tube EtOH: 94.12% Tube product: 5.88% 10%: Tube EtOH: 87.53% Tube product: 12.47% |
| EV06028 | 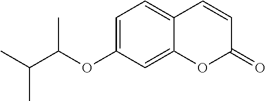 | 93% | 232.11 | 3.29 | 20%: Tube EtOH: 94.21% Tube product: 5.79% 10%: Tube EtOH: 86.74% Tube product: 13.26% |
| EV06036 | 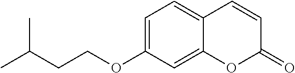 | 65% | 232.11 | 3.24 | 20%: Tube EtOH: 86.63% Tube product: 13.73% 10%: Tube EtOH: 86.10% Tube product: 13.90% |
| EV06044 | 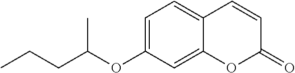 | 91% | 232.11 | 3.37 | 20%: Tube EtOH: 96.12% Tube product: 3.82% 10%: Tube EtOH: 83.91% Tube product: 16.08% |
| EV06056 | 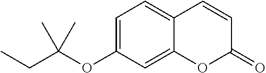 | — | 232.11 | 3.20 | — |
| EV06062 | 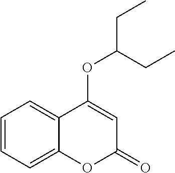 | 87% | 232.11 | 2.96 | 20%: Tube EtOH: 98.26% Tube product: 1.74% 10%: Tube EtOH: 93.10% Tube product: 6.90% 5%: Tube EtOH: 96.08% Tube product: 3.20% 1%: Tube EtOH: 87.08% Tube product: 12.92% |
| EV06068 | 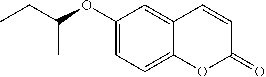 | 79% | 218.09 | 2.92 | 20%: Tube EtOH: 93.85% Tube product: 6.15% 10%: Tube EtOH: 90.91% Tube product: 9.09% |
| EV06072 | 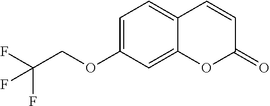 | — | 244.17 | 2.58 | — |
| EV06086 EV06094 | 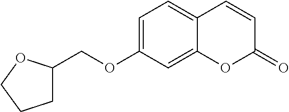 | — | 246.27 | 2.04 | — |
| EV06088 | 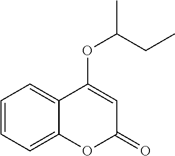 | 100% | 218.09 | 2.44 | 20%: Tube EtOH: 91.04% Tube product: 8.96% 10%: Tube EtOH: 90.50% Tube product: 9.50% |
| EV06098 | 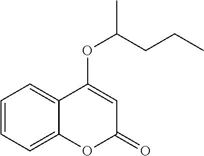 | 91% | 232.11 | 2.88 | 20%: Tube EtOH: 97.57% Tube product: 2.43% 10%: Tube EtOH: 79.29% Tube product: 20.71% |
| EV06110 | 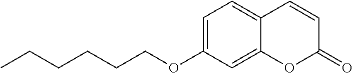 | 95% | 246.13 | 3.84 | 20%: Tube EtOH: 89.06% Tube product: 10.94% 10%: Tube EtOH: 91.09% Tube product: 8.91% |
| EV06128 | 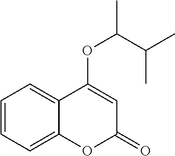 | 84% | 232.11 | 2.80 | 20%: Tube EtOH: 66.83% Tube product: 33.17% |
| EV06136 | 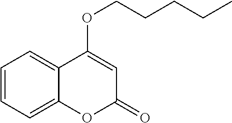 | 100% | 232.11 | 2.91 | 20%: Tube AcOEt: 88.56% Tube product: 11.44% |
| EV06140 | 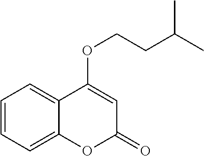 | 91% | 232.11 | 2.75 | 20%: Tube EtOH: 91.92% Tube product: 8.08% 10%: Tube EtOH: 90.10% Tube product: 9.90% |
| EV06144 | 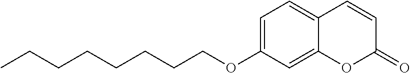 | 100% | 274.35 | 4.73 | 20%: Tube EtOH: 91.73% Tube product: 8.37% 10%: Tube EtOH: 90.00% Tube product: 10.00% 5%: Tube EtOH: 91.35% Tube product: 8.65% 1%: Tube EtOH: % Tube product: % |
| EV06148 | 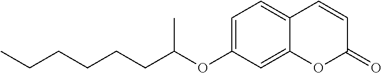 | 93% | 274.35 | 4.70 | 20%: Tube EtOH: 96.00% Tube product: 4.00% 10%: Tube EtOH: 93.66% Tube product: 6.34% |
| EV06154 | 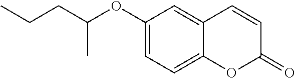 | 91% | 232.11 | 3.37 | 20%: Tube EtOH: 96.57% Tube product: 3.43% 10%: Tube EtOH: 93.03% Tube product: 6.97% |
| EV06162 | 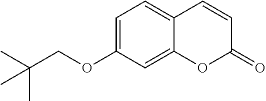 | 49% | 232.11 | 3.25 | In progress |
| EV06188 | 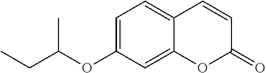 | In pro- gress | 218.09 | 2.92 | In progress |

TABLE 3
Coumarin ethers- repulsive indices

| | | | Re- | | |
|---|---|---|---|---|---|
| Reaction | | Mol. | pulsive | Tested | Khideux |
| number | Ether | Mass | Index | Concentration | (p-value) |
| EV04036 | 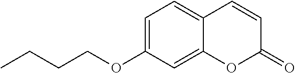 | 218.09 | 0 | 0.905 mmol/mL | nd (2.720E−14) |
| EV04032 | 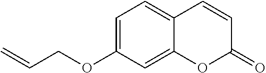 | 202.06 | 0.186 0.245 | 0.977 mmol/mL | 18.993 (0.001) 26.926 (2.058E−5) |
| EV04054 | 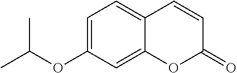 | 204.08 | 0.655 | 0.967 mmol/mL | 11.270 (0.024) |
| EV04058 | 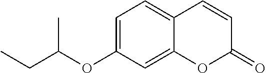 | 218.09 | 0.760 0.824 | 0.905 mmol/mL 0.404 mmol/mL | 28.825 (0.0003) nd (4.176E−10) |
| EV04070 | 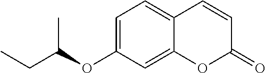 | 218.09 | 0.827 0.856 | 0.905 mmol/mL 0.404 mmol/mL | 14.171 (0.077) 3.844 (0.427) |
| EV04084 | 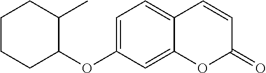 | 258.12 | −0.010 | 0.765 mmol/mL | nd (5.110E−13) |
| EV04090 | 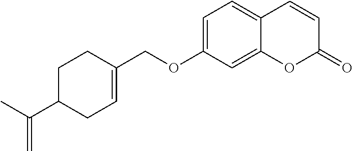 | 296.14 | 0.404 | 0.666 mmol/mL | 10.783 (0.029) |
| EV04094 | 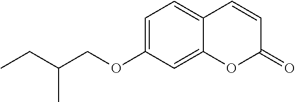 | 232.11 | 0.729 0.793 | 0.850 mmol/mL 0.382 mmol/mL | 9.819 (0.278) 35.618 (8.556E−6) |
| EV04114 | 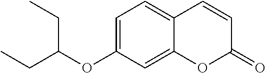 | 232.11 | 0.862 0.760 | 0.850 mmol/mL 0.382 mmol/mL | 29.343 (0.0003) 31.355 (2.591E−6) |
| EV04188 | 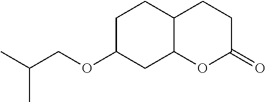 | 226.16 | 0.647 | 0.873 mmol/mL | 12.280 (0.015) |
| EV05184 | 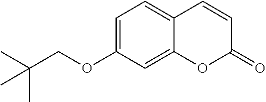 | 232.11 | — | — | — |
| EV06018 | 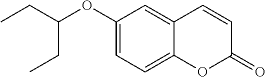 | 232.11 | 0.430 0.621 | 0.850 mmol/mL | 24.870 (5.342E−5) nd (1.392E−12) |
| EV06026 | 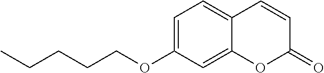 | 232.11 | 0.882 0.751 | 0.850 mmol/mL 0.382 mmol/mL | 21.153 (0.0003) 32.124 (8.848E−5) |
| EV06028 | 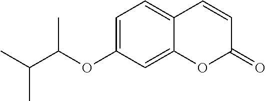 | 232.11 | 0.884 0.735 | 0.850 mmol/mL 0.382 mmol/mL | nd (4.702E−12) 13.504 (0.009) |
| EV06036 | 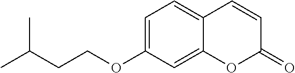 | 232.11 | 0.733 0.722 | 0.850 mmol/mL 0.382 mmol/mL | 15.720 (0.003) 22.740 (0.002) |
| EV06044 | 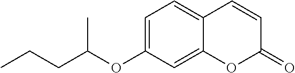 | 232.11 | 0.922 0.678 | 0.850 mmol/mL 0.382 mmol/mL | 2.611 (0.978) 34.480 (5.939E−7) |
| EV06056 | 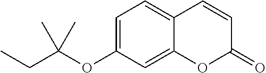 | 232.11 | — | — | — |
| EV06062 | 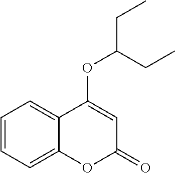 | 232.11 | 0.965 0.862 0.936 0.742 | 0.850 mmol/mL 0.382 mmol/mL 0.181 mmol/mL 0.056 mmol/mL | 12.825 (0.171) 10.446 (0.034) 27.870 (1.324E−5) 20.300 (0.001) |
| EV06068 | 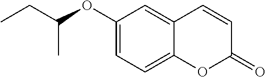 | 218.09 | 0.877 0.818 | 0.905 mmol/mL 0.404 mmol/mL | 7.950 (0.093) 15.592 (0.003) |
| EV06072 | 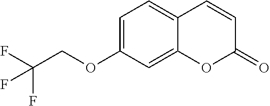 | 244.17 | — | — | — |
| EV06086 EV06094 | 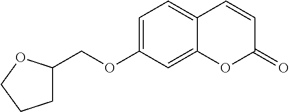 | 246.72 | — | — | — |
| EV06088 | 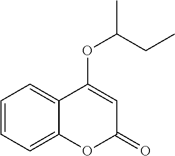 | 218.09 | 0.821 0.810 | 0.905 mmol/mL 0.404 mmol/mL | 13.384 (0.010) 13.006 (0.011) |
| EV06098 | 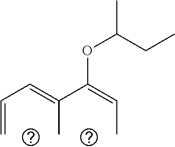 | 232.11 | 0.951 0.586 | 0.850 mmol/mL 0.382 mmol/mL | 8.043 (0.090) 9.967 (0.041) |
| EV06110 | 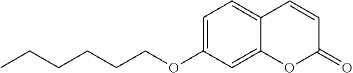 | 246.13 | 0.781 0.822 | 0.802 mmol/mL 0.360 mmol/mL | nd (1.097E−10) 5.038 (0.283) |
| EV06128 | 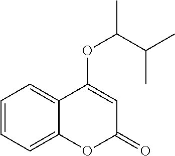 | 232.11 | 0.337 | 0.850 mmol/mL | nd (1.211E−11) |
| EV06136 | 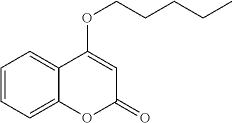 | 232.11 | 0.771 | 0.850 mmol/mL | 12.379 (0.015) |
| EV06140 | 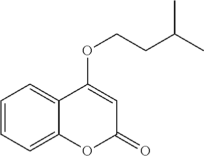 | 232.11 | 0.838 0.802 | 0.850 mmol/mL 0.382 mmol/mL | 13.346 (0.009) 2.765 (0.598) |
| EV06144 | 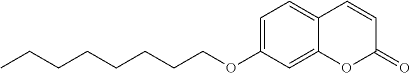 | 274.35 | 0.833 0.800 0.827 | 0.719 mmol/mL 0.323 mmol/mL 0.153 mmol/mL | 12.379 (0.015) nd (5.826E−8) 15.826 (0.003) |
| EV06148 | 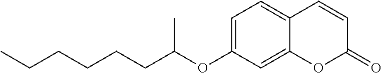 | 274.35 | 0.920 | 0.719 mmol/mL | 6.275 (0.015) |
| EV06154 | 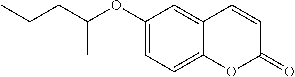 | 232.11 | 0.931 | 0.850 mmol/mL | 14.875 (0.005) |

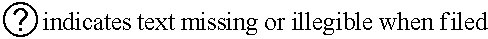

### Etherification of 7-Hydroxycoumarin: Mitsunobu Reaction

TABLE 4
7-hydroxycoumarin derivatives produced via Mitsunobu reaction

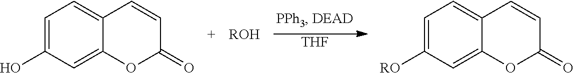

| Reaction | | Isolated | Molecular | |
|---|---|---|---|---|
| number | Ether | yield | Mass | LogP |
| EV06110 | 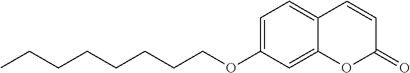 | 95% | 246.13 | 3.84 |
| EV06144 | 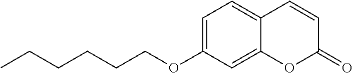 | 100% | 274.35 | 4.73 |
| EV06148 | 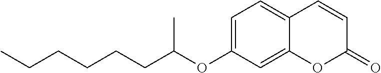 | 93% | 274.35 | 4.70 |
| EV06188 | 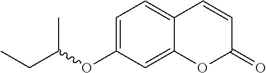 | 100% | 218.09 | 2.92 |
| EV06018 | 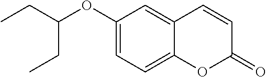 | 79% | 232.11 | 3.44 |
| EV06068 | 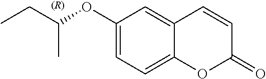 | 79% | 218.09 | 2.92 |
| EV06154 | 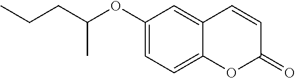 | 91% | 232.11 | 3.37 |

Williamson reaction was used to make 7-hydroxycoumarin derivatives not obtainable via Mitsunobu:

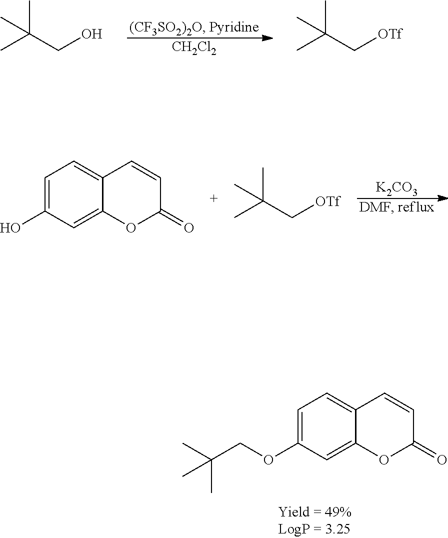

The 7-hydroxycoumarin analogues including EV04114, EV04070, and EV06044 have shown significant repellent activity against drosophila.

TABLE 5
Etherification of 4-hydroxycoumarin

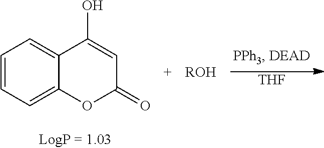

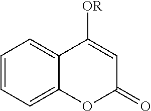

| | | Iso- | Mole- | |
|---|---|---|---|---|
| Reaction | | lated | cular | |
| number | Ether | yield | Mass | LogP |
| EV06062 | 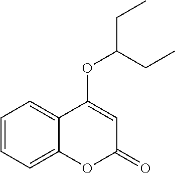 | 87% | 232.11 | 2.96 |
| EV06088 | 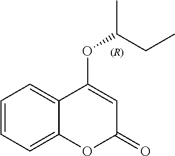 | 100% | 218.09 | 2.44 |
| EV06098 | 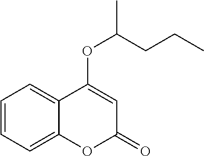 | 91% | 232.11 | 2.88 |
| EV06128 | 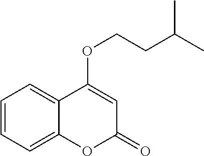 | 84% | 232.11 | 2.80 |
| EV06136 | 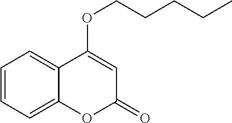 | 100% | 232.11 | 2.91 |
| EV06140 | 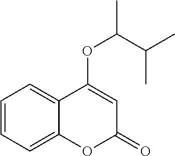 | 91% | 232.11 | 2.75 |

### Example 2

### Coumarin Amide Derivatives

Coumarin was then modified at the 4 or 7 carbon as represented by the following compounds:

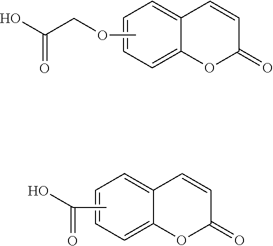

Reactions included:

TABLE 6
Amidated coumarin derivatives

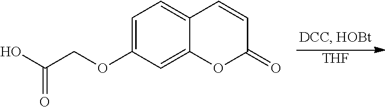

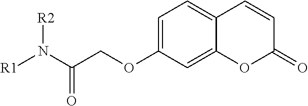

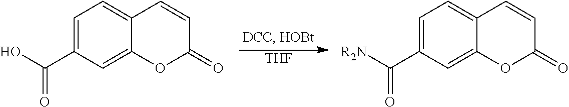

| Reaction | | Isolated | Molecular | |
|---|---|---|---|---|
| number | Amide | yield | mass | LogP |
| EV06178 | 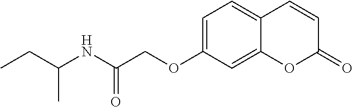 | 68% | 275.11 | 1.82 |
| EV06020 | 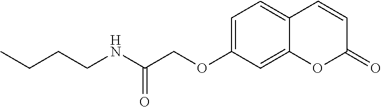 | 42% | 275.11 | 1.84 |
| EV06166 | 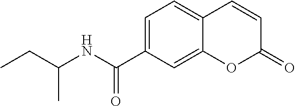 | 58% | 245.27 | 2.15 |
| EV07038 | 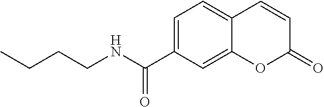 | 37% | 245.27 | 2.18 |

Amidification at carbon 4 was accomplished via the following steps: 1) Etherification of 4-hydroxycoumarin by methyl glycolate; 2) saponification; and amidification.

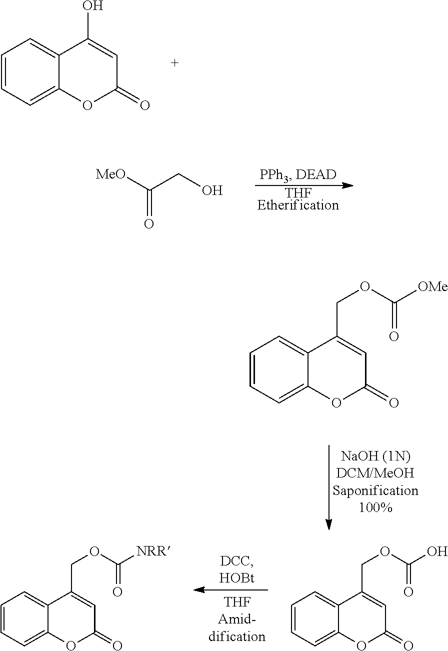

The methyl glycolate coumarin proved difficult to isolate, so other reaction schema were explored.

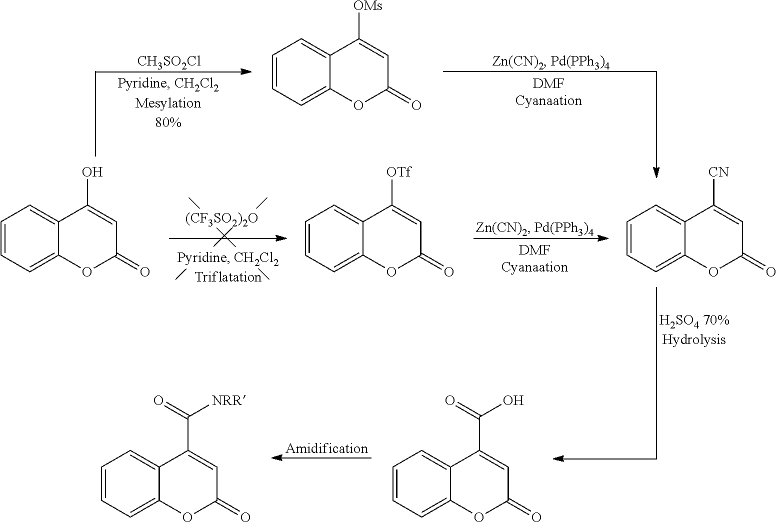

The 4 triflated coumarin was not stable enough to be isolated. For this reason, mesylation was instead carried out.

TABLE 7
Additional amidated coumarin derivatives

| Reaction | | Isolated | Mol. | | |
|---|---|---|---|---|---|
| number | Amide | yield | Mass | LogP | In vivo tests |
| EV05084 | 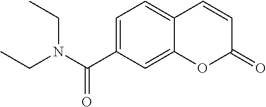 | 100% | 245.27 | 1.79 | Tube EtOH: 65.03% Tube product: 34.97% |
| EV05138 | 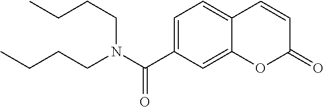 | 81% | 301.38 | 3.73 | 20%: Tube EtOH: 86.27% Tube product: 13.73% 10%: Tube EtOH: 82.54% Tube product: 17.46% |
| EV06096 | 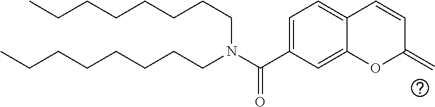 | 58% | 413.29 | 7.29 | 20%: Tube EtOH: 95.33% Tube product: 4.67% 10%: Tube EtOH: 67.84% Tube product: 32.16% |
| EV05118 | 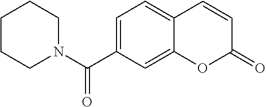 | 45% | 257.10 | 1.93 | Tube EtOH: 44.83% Tube product: 55.17% |
| EV05144 | 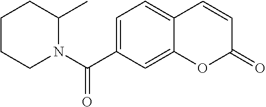 | 76% | 271.31 | 2.35 | Tube EtOH: 80.10% Tube product: 19.90% |
| EV06166 | 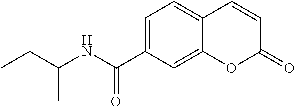 | 58% | 245.27 | 2.15 | Tube EtOH: 62.58% Tube product: 37.42% |

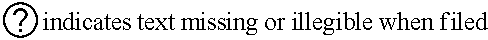

TABLE 8
Amidated coumarin derivatives- repulsive indices

| Reaction | | Mol. | | Tested | Khideux |
|---|---|---|---|---|---|
| number | Amide | Mass | RI | Concentration | (p-value) |
| EV05084 | 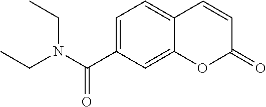 | 245.27 | 0.301 | 0.805 mmol/mL | 10.757 (0.029) |
| EV05138 | 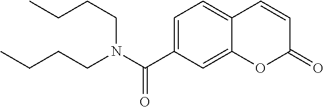 | 301.38 | 0.722 0.650 | 0.655 mmol/mL 0.294 mmol/mL | 12.670 (0.013) nd (9.735E−8) |
| EV06096 | 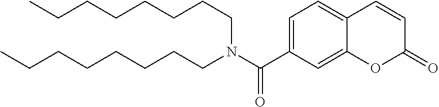 | 413.29 | 0.907 0.357 | 0.477 mmol/mL 0.233 mmol/mL | 5.187 (0.269) 34.671 (5.427E−7) |
| EV05118 | 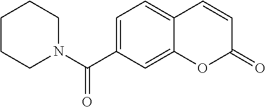 | 257.10 | −0.103 | 0.768 mmol/mL | nd (3.583E−12) |
| EV05144 | 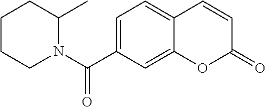 | 271.31 | 0.599 | 0.727 mmol/mL | 10.806 (0.029) |
| EV06166 | 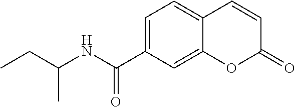 | 245.27 | 0.251 | 0.850 mmol/mL | 25.980 (9.632E−6) |

TABLE 9
Amidated coumarin derivatives

| Reaction | | Isolated | Mol. | | |
|---|---|---|---|---|---|
| number | Amide | yield | Mass | LogP | In vivo tests |
| EV05096 | 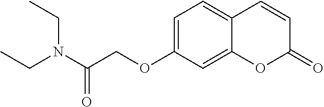 | 69% | 275.11 | 1.46 | Tube EtOH: 90.11% Tube product: 9.89% |
| EV05120 | 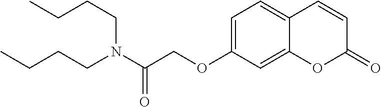 | 96% | 331.18 | 3.39 | 20%: Tube EtOH: 85.58% Tube product: 14.42% 10%: Tube EtOH: 90.36% Tube product: 9.64% 1.032 mmol/mL: Tube EtOH: 96.89% Tube product: 3.11% |
| EV06092 | 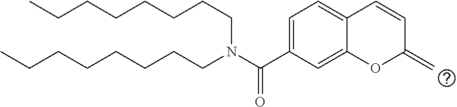 | 94% | 443.30 | 6.95 | Tube EtOH: 67.66% Tube product: 32.34% |
| EV05088 | 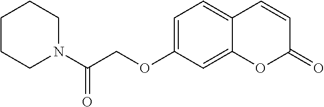 | 45% | 287.31 | 1.59 | Tube EtOH: 53.40% Tube product: 46.60% |
| EV05134 | 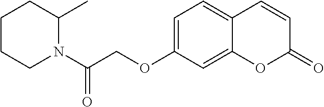 | 79% | 301.34 | 2.01 | Tube EtOH: 88.94% Tube product: 11.06% Tube EtOH: 62.05% Tube product: 37.95% Tube EtOH: 72.41% Tube product: 27.59% |
| EV06178 | 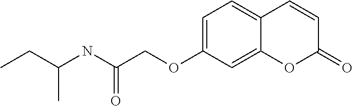 | In progress | 275.11 | 1.82 | In progress |

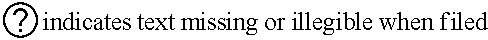

TABLE 10
Aminated coumarin derivatives- RI

| Reaction | | Mol. | | Tested | Khideux |
|---|---|---|---|---|---|
| number | Amide | Mass | RI | Concentration | (p-value) |
| EV05096 | 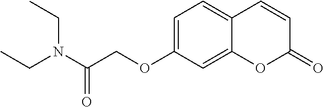 | 275.11 | 0.802 | 0.717 mmol/mL | 18.256 (0.032) |
| EV05120 | 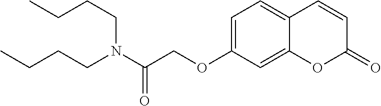 | 331.18 | 0.708 0.807 0.938 | 0.596 mmol/mL 0.268 mmol/mL 1.032 mmol/mL | nd (1.339E−09) nd (4.363E−10) 2.297 (0.513) |
| EV06092 | 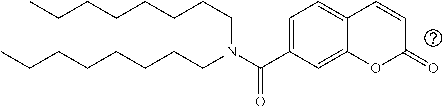 | 443.30 | 0.353 | 0.445 mmol/mL | 16.608 (0.002) |
| EV05088 | 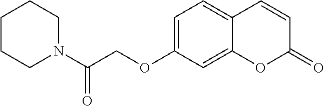 | 287.31 | 0.068 | 0.687 mmol/mL | 28.447 (1.012E−05) |
| EV05134 | 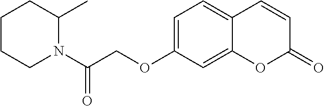 | 301.34 | 0.775 0.240 0.448 | 0.654 mmol/mL | 5.496 (0.240) nd (5.734E−16) 29.788 (5.407E−6) |

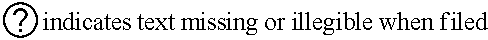

### Example 3

### Open Coumarin Derivatives

TABLE 11
Open coumarin derivatives

| | | Iso- | | | |
|---|---|---|---|---|---|
| Reaction | | lated | Mol. | | In vivo |
| number | Molecular | yield | Mass | LogP | tests |
| — | 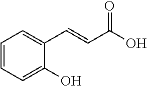 | — | 164.05 | 1.83 | Tube EtOH: 52.43% Tube product: 47.57% |
| EV05056 | 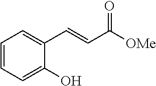 | 56% | 178.18 | 2.21 | Tube EtOH: 70.10% Tube product: 29.90% |

TABLE 12
Open coumarin derivatives- RI

| Reaction | | Mol. | | Tested | Khideux |
|---|---|---|---|---|---|
| number | Molecular | Mass | RI | Concentration | (p-value) |
| — | 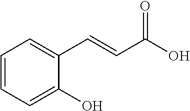 | 164.05 | 0.049 | 1.203 mmol/mL | nd (3.253E−08) |
| EV05056 | 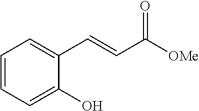 | 178.18 | 0.402 | 1.107 mmol/mL | 24.743 (5.667E−05) |

### Example 4

### Esterified Coumarin Derivatives

TABLE 13
Esterified coumarin derivatives

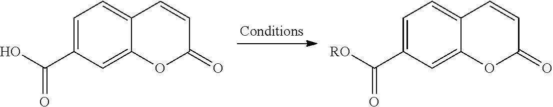

| Reaction | | Isolated | Molecular | | |
|---|---|---|---|---|---|
| number | Ester | yield | Mass | LogP | In vivo tests |
| EV05162 | 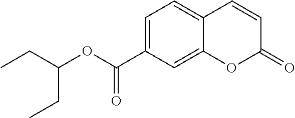 | 60% | 260.28 | 3.44 | Tube EtOH: 81.22% Tube product: 18.78% Tube EtOH: 63.18% Tube product: 36.82% Tube EtOH: 67.86% Tube product: 32.14% |
| EV05178 EV06048 | 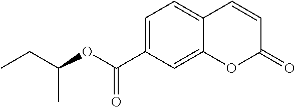 | 66% | 246.09 | 3.08 | 20%: Tube EtOH: 85.50% Tube product: 14.50% 10%: Tube EtOH: 54.77% Tube product: 45.23% |

TABLE 14
Esterified coumarin derivatives- RI

| Reaction | | Molecular | | Tested | Khideux |
|---|---|---|---|---|---|
| number | Ester | Mass | RI | Concentration | (p-value) |
| EV05162 | 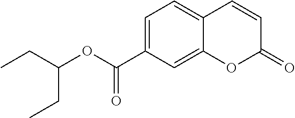 | 260.28 | 0.624 0.264 0.357 | 0.758 mmol/mL | 6.591 (0.159) 33.447 (9.675E−7) 21.990 (0.0002) |
| EV05178 EV06048 | 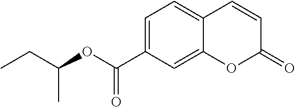 | 246.09 | 0.710 0.095 | 0.802 mmol/mL 0.360 mmol/mL | 15.843 (0.003) nd (9.751E−16) |

TABLE 15
Esterified coumarin derivates

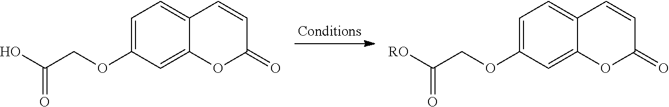

| Reaction | | Isolated | Mol. | | |
|---|---|---|---|---|---|
| number | Ester | yield | Mass | LogP | In vivo tests |
| EV05174 | 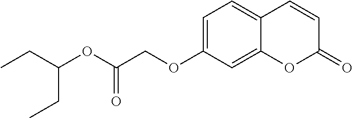 | 60% | 260.28 | 3.07 | 20%: Tube EtOH: 84.31% Tube product: 15.69% 10%: Tube EtOH: 68.29% Tube product: 31.71% |
| EV06046 | 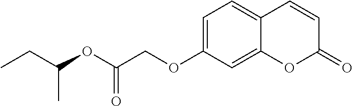 | 68% | 276.10 | 2.55 | Tube EtOH: 55.22% Tube product: 44.78% Tube EtOH: 52.24% Tube product: 47.76% |
| EV06058 | 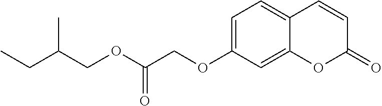 | 53% | 290.32 | 2.94 | Tube EtOH: 75.74% Tube product: 24.26% |

TABLE 16
Esterified coumarin derivatives- RI

| Reaction | | Mol. | Repulsive | Tested | Khideux |
|---|---|---|---|---|---|
| number | Ester | Mass | Index | Concentration | (p-value) |
| EV05174 | 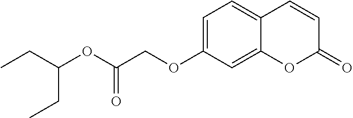 | 260.28 | 0.686 0.366 | 0.680 mmol/mL 0.341 mmol/mL | 16.265 (0.003) nd (3.800E−13) |
| EV06046 | 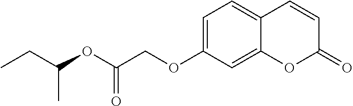 | 276.10 | 0.104 0.045 | 0.715 mmol/mL | nd (4.188E−23) nd (5.315E−12) |
| EV06058 | 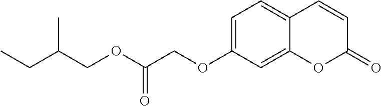 | 290.32 | 0.515 | 0.680 mmol/mL | nd (1.418E−8) |

TABLE 17
Esterified coumarin derivatives

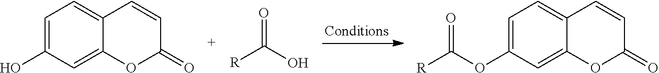

| Reaction | | Isolated | Molecular | | |
|---|---|---|---|---|---|
| number | Ester | yield | Mass | LogP | In vivo tests |
| EV04016 | 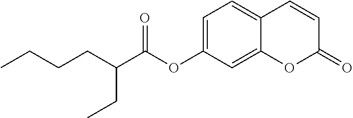 | 86% | 288.14 | 4.41 | Tube EtOH: 55.50% Tube product: 44.50% |
| EV04024 | 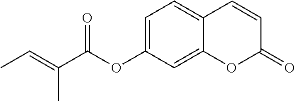 | 83% | 244.07 | 3.16 | Tube EtOH: 62.94% Tube product: 37.06% |
| EV04030 | 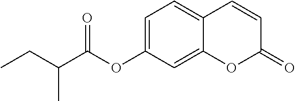 | 82% | 246.09 | 3.08 | Tube EtOH: 44.80% Tube product: 55.20% |
| EV04062 | 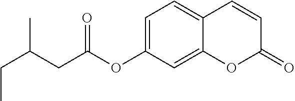 | 56% | 260.10 | 3.27 | Tube EtOH: 51.90% Tube product: 48.10% |
| EV04122 | 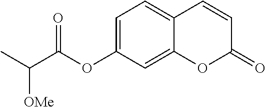 | 53% 30% | 276.06 | 1.58 | Tube EtOH: 31.82% Tube product: 68.18% |

TABLE 17
Esterified derivatives- RI

| Reaction | | | | Tested | Khideux |
|---|---|---|---|---|---|
| number | Ester | Mol. Mass | RI | Concentration | (p-value) |
| EV04016 | 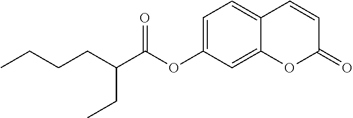 | 288.14 | 0.110 | 0.685 mmol/mL | 31.624 (2.284E−06) |
| EV04024 | 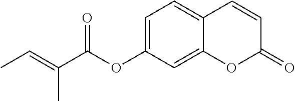 | 244.07 | 0.259 | 0.809 mmol/mL | nd (5.006E−09) |
| EV04030 | 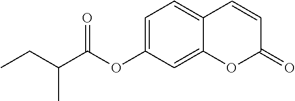 | 246.09 | −0.103 | 0.802 mmol/mL | nd 91.135E−10) |
| EV04062 | 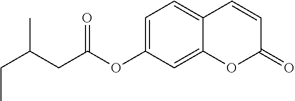 | 260.10 | 0.038 | 0.759 mmol/mL | nd (9.714E−14) |
| EV04122 | 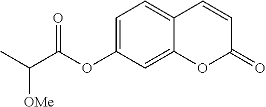 | 276.06 | −0.364 | 0.715 mmol/mL | 33.771 (8.301E−07) |

### Example 5

### Tests In Vivo on Drosophila

Description of Test system. To evaluate repellent efficacy, compounds were added to test chambers, each containing 40 drosophila. The test was performed in 3 phases: 1) fast (flies aspirated to test tubes); 2) implementation of the system (addition of filter paper, addition of solvent containing test compound); and 3) counting of the flies (after 22 hours exposure to test compound). The number of drosophila in each tube were counted: number of flies in the “filter paper+Solvent” tube was designated N1 and the number of flies in the “filter paper impregnated with test compound” was designated N2.

The Repulsive Index (R1) is given by the following equation: (N1−N2)/(N1+N2).

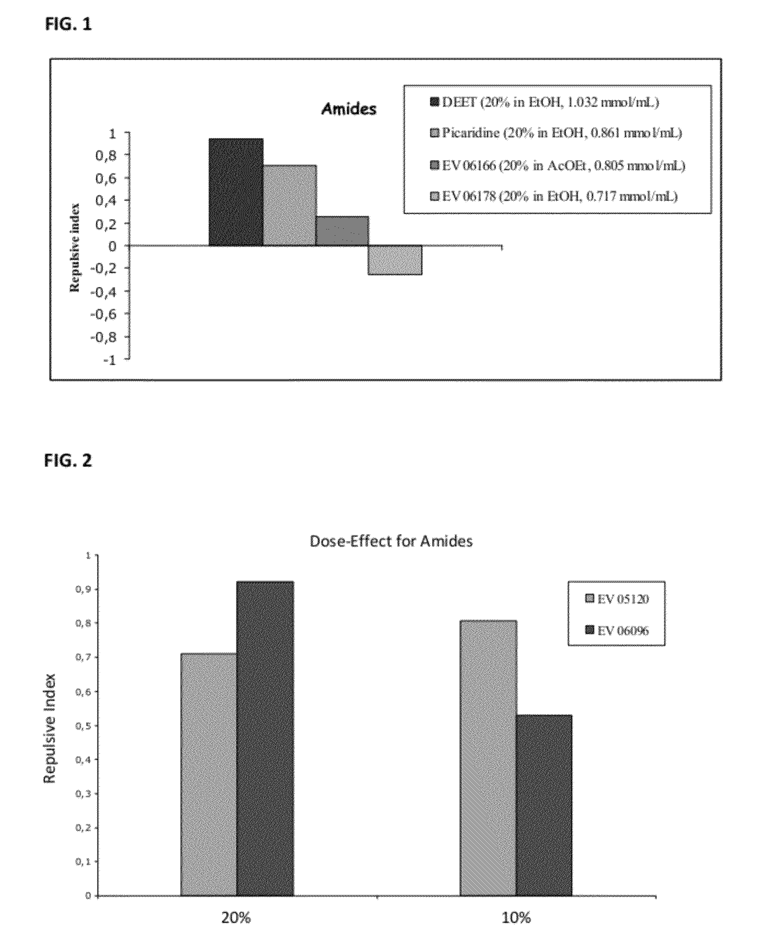

Surprisingly, some coumarin derivatives were experimentally determined to be more “attractive” than “repellent” (i.e. the compounds were associated with a negative RI). This observation suggests that a skilled person would not be able to predict ahead of time whether a specific coumarin derivative, or class of derivative (e.g. secondary amides) would be an effective repellent. For example, when subjected to the above-described repellency test, the RI against drosophila for EV06166 (RI˜0.2) and EV06178 (RI˜−0.2) were found to be significantly lower as compared to the RI's of two known repellents: DEET (RI>0.9) and picaridine (RI>0.6) (FIG. 1). Inventors concluded secondary amides may not provide a significant repellent activity, and further, EV06178 appeared to provide attractant activity.

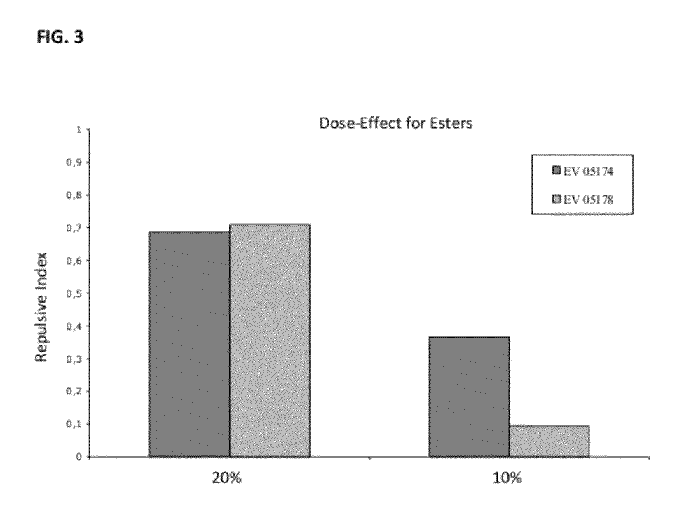

In contrast, other amides (FIG. 2) exhibited repellent indices greater than picaridine and similar to DEET. Both 20% and 10% solutions in EtOH of EV05120 and EV06096 were subjected to the repellency test described above. The data indicated both compounds acted as repellents, and that even when diluted 2-fold, the EV05120 appeared to retain repellency (RI=˜0.7 at 20% and ˜0.8 at 10%).

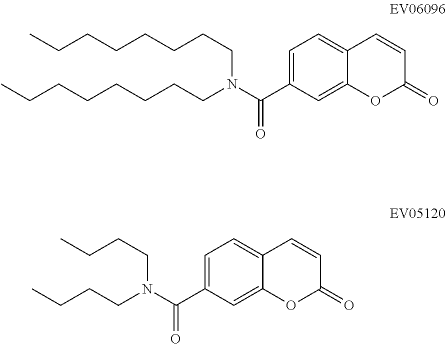

Various esterified coumarin derivatives were also prepared and tested according to the method described above. Solutions of 10% and 20% in EtOH of and were determined to have the respective R1 values: EV05174 (˜0.7 at 20%; ˜0.35 at 10%), EV05178 (˜0.7 at 20%, ˜0.07 at 10%) (FIG. 3). These data indicate a skilled person could not predict ahead of time what will be an effective concentration for a given species or genus of coumarin derivative. In this case, two related coumarin esters exhibit significantly different effective repellent concentrations.

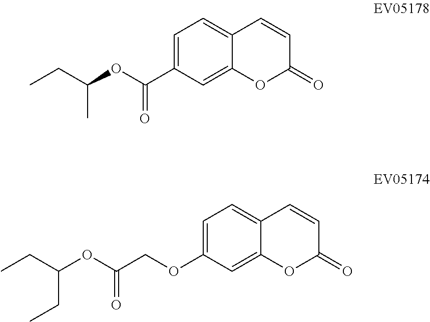

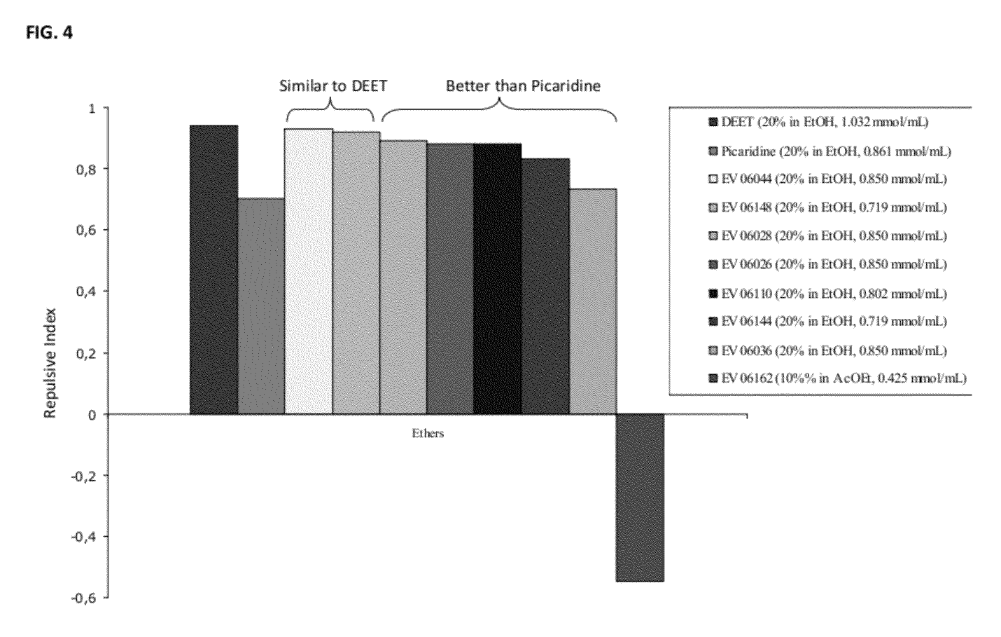

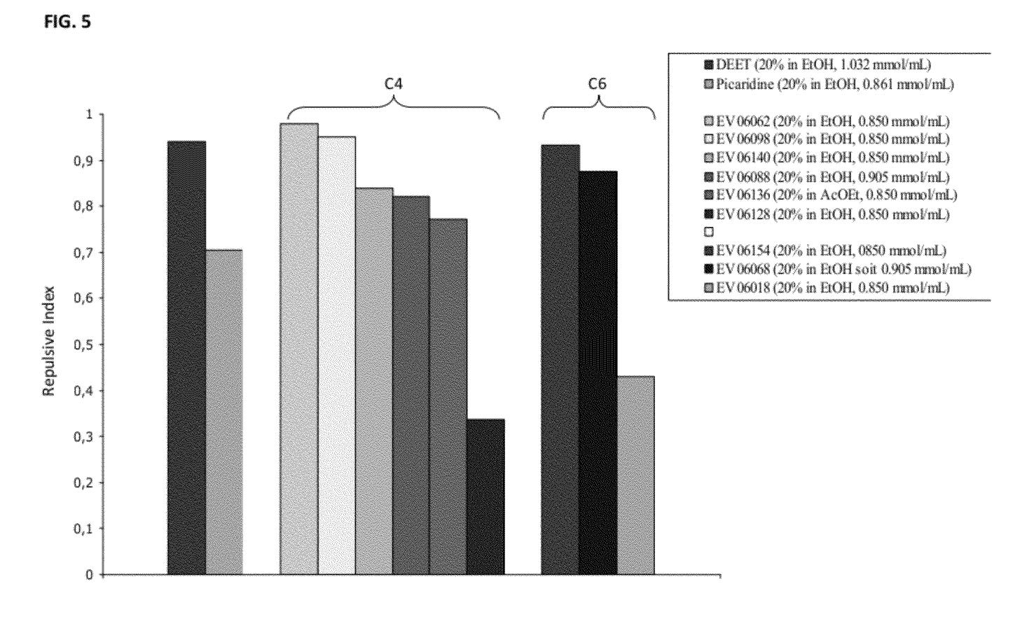

Various ethers were also tested (FIG. 4, Tables 2 and 3), including EV06044, EV06148, EV06028, EV06026, EV06110, EV06144, EV06036, and EV06162. Surprisingly, 2 compounds had RIs comparable to DEET, 5 compounds had RIs superior to picaridine, and one compound (EV06162) appeared to be a strong drosophila attractant. The results of tests performed on C4 and C6 ethers are presented in FIG. 5. In this experiment, 3 compounds presented similar repellent activity as DEET and 3 compounds has a better repellent activity than picaridine at 20% in EtOH.

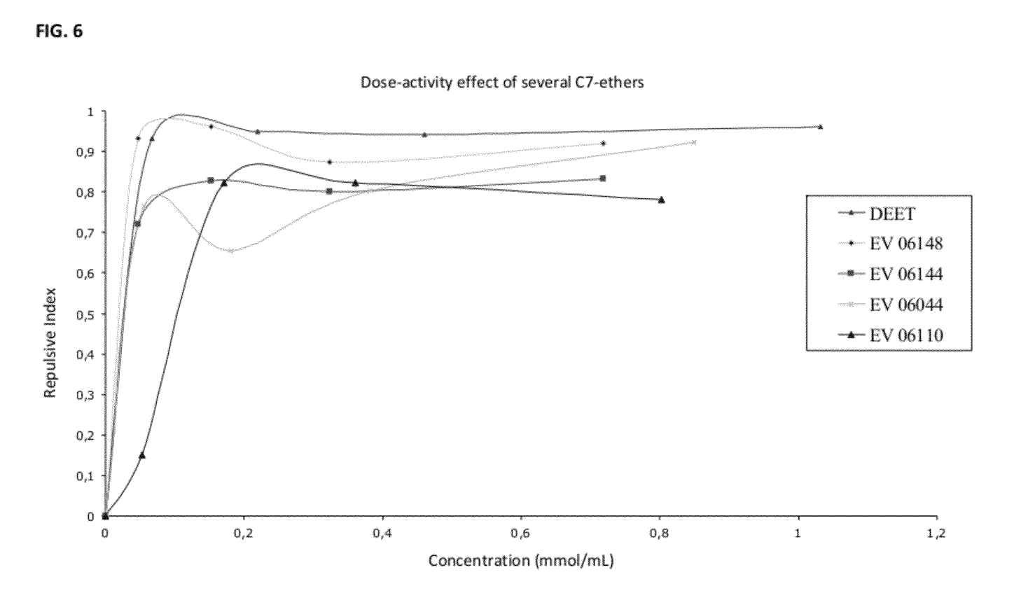

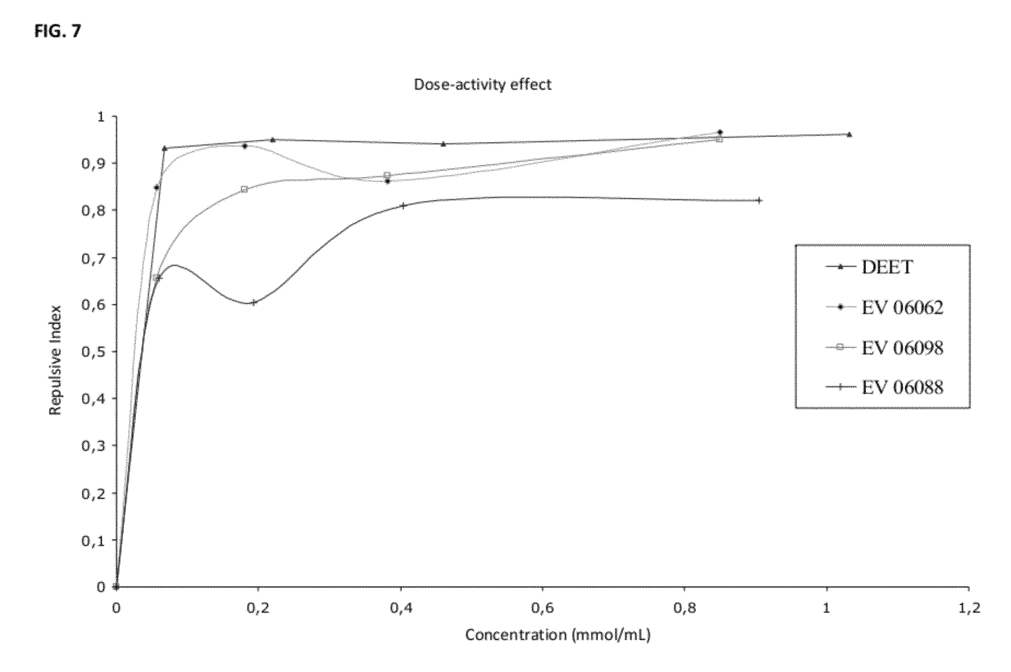

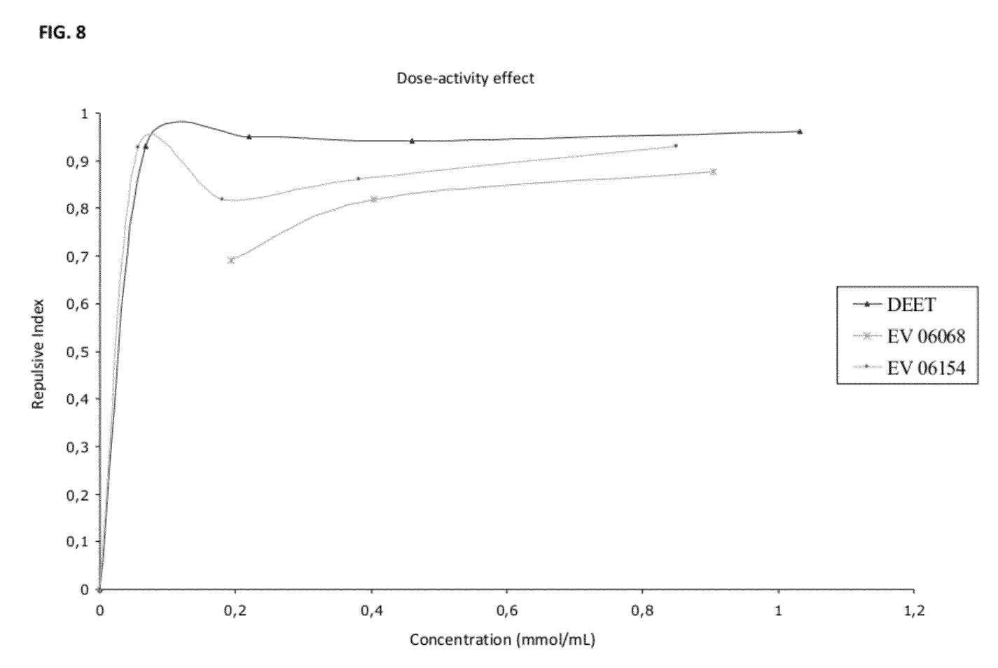

To examine the dose effect for several C7-ethers, EV06148, EV06144, EV06044, and EV05110 were all evaluated at 1%, 5%, 10%, and 20% in EtOH (FIG. 6). The data indicate, in part, EV06148 remained as effective a repellent as DEET even at the 1% concentration level. The same study (1%, 2% . . . in EtOH) was performed on several C4-ethers (FIG. 7), and these data indicate, in part, EV06062 remains as good a repellent as DEET even at the 1% concentration. The results of the same test run on C6-ethers are presented in FIG. 8. EV06154 performed effectively as a repellent at the 1% concentration, similar to DEET.

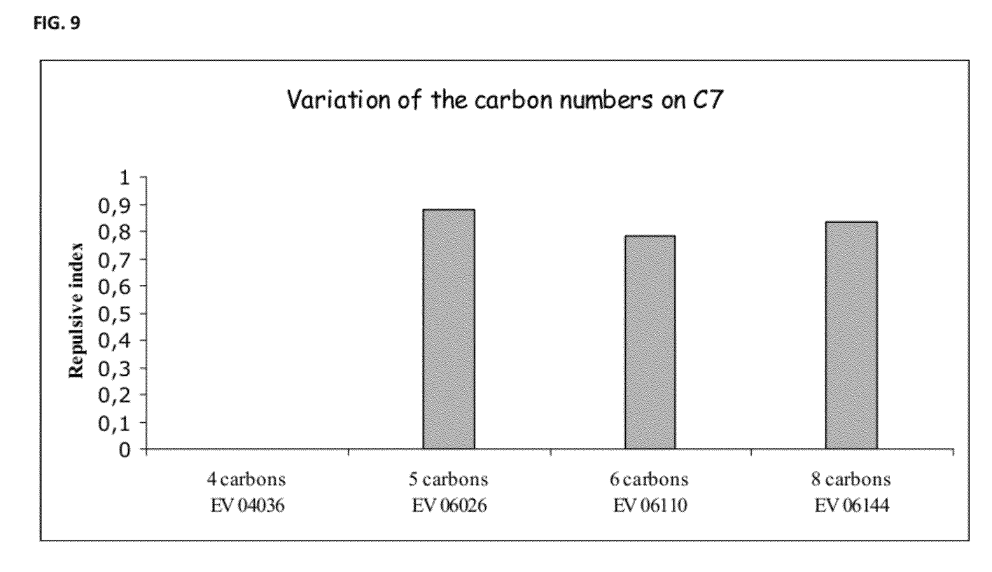

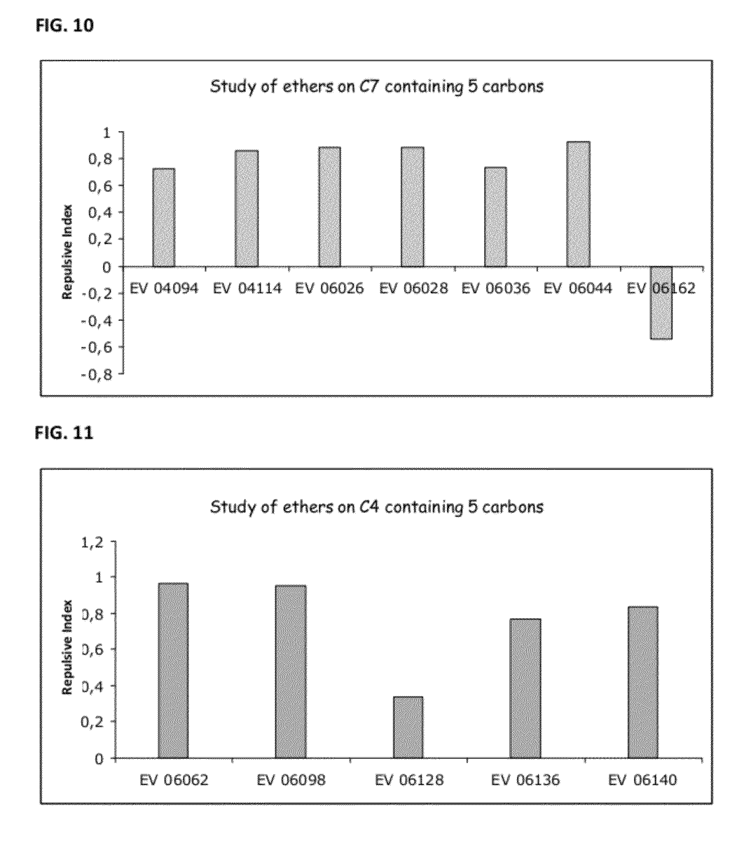

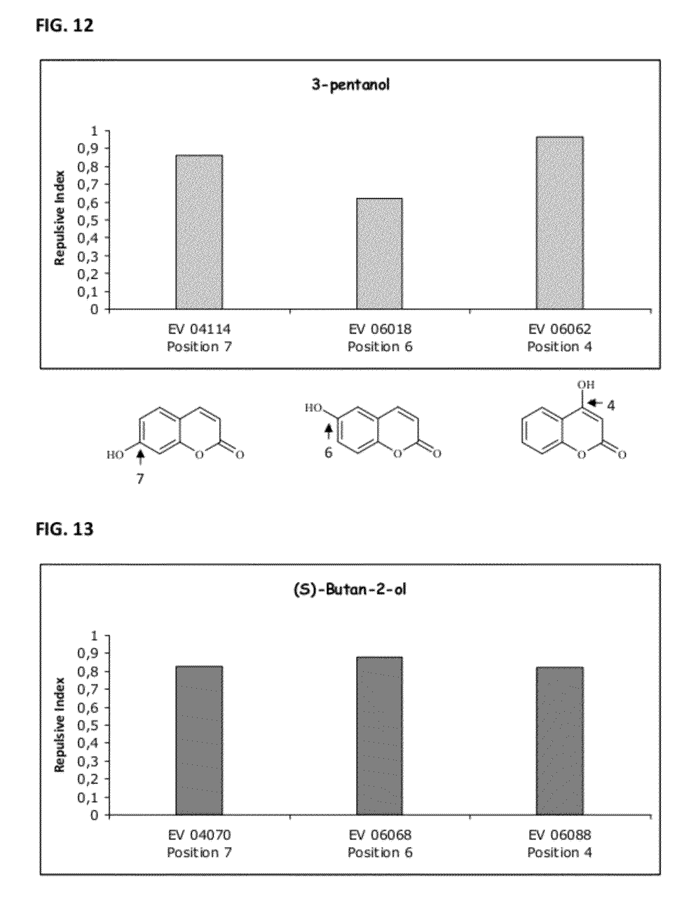

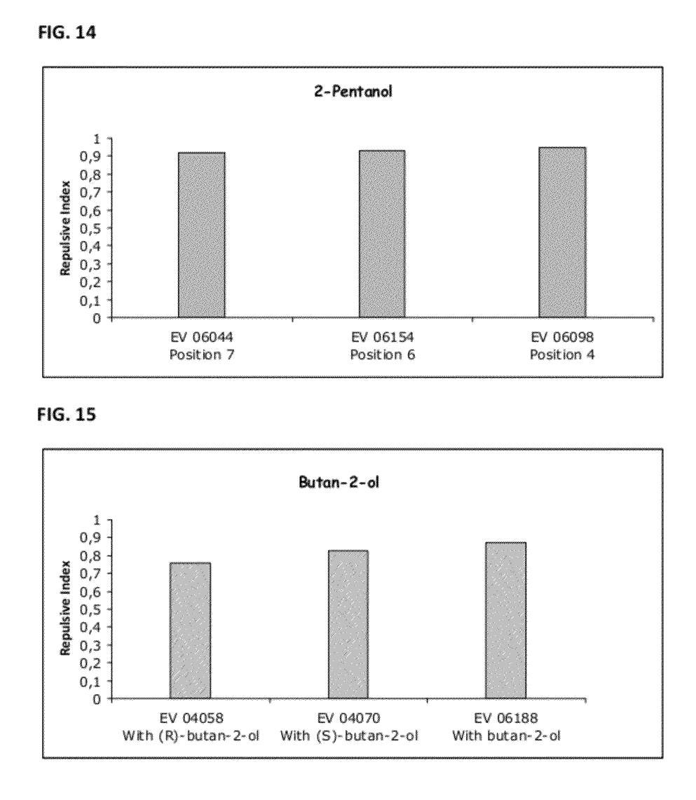

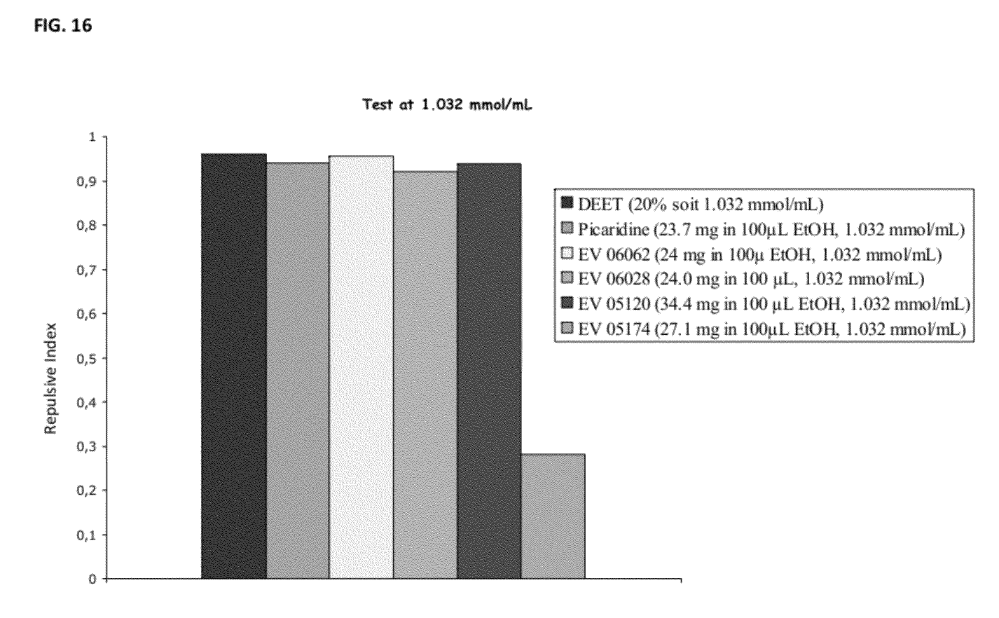

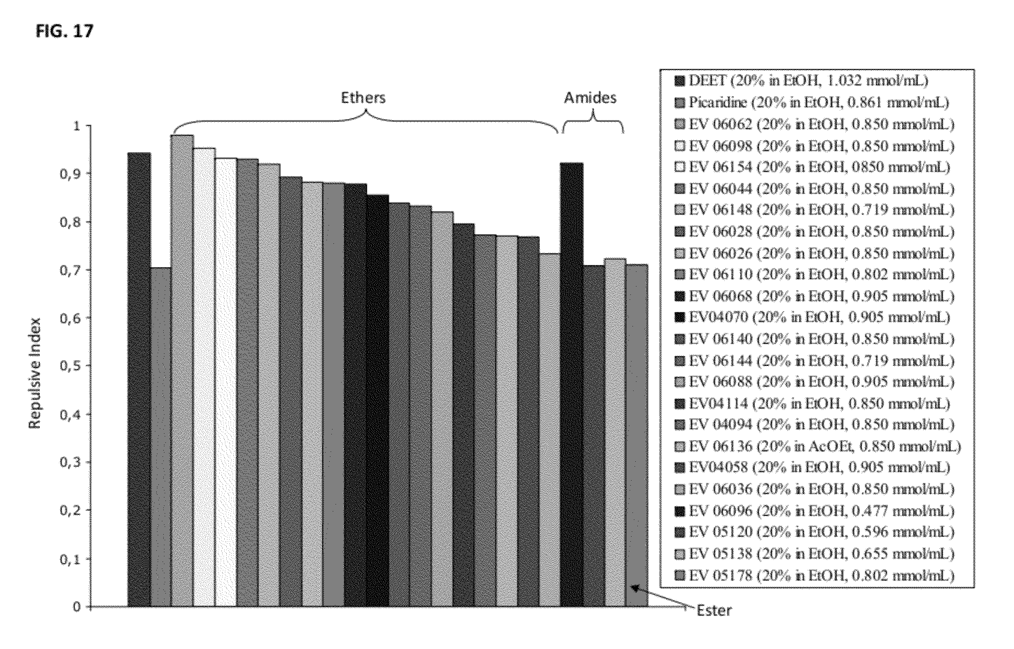

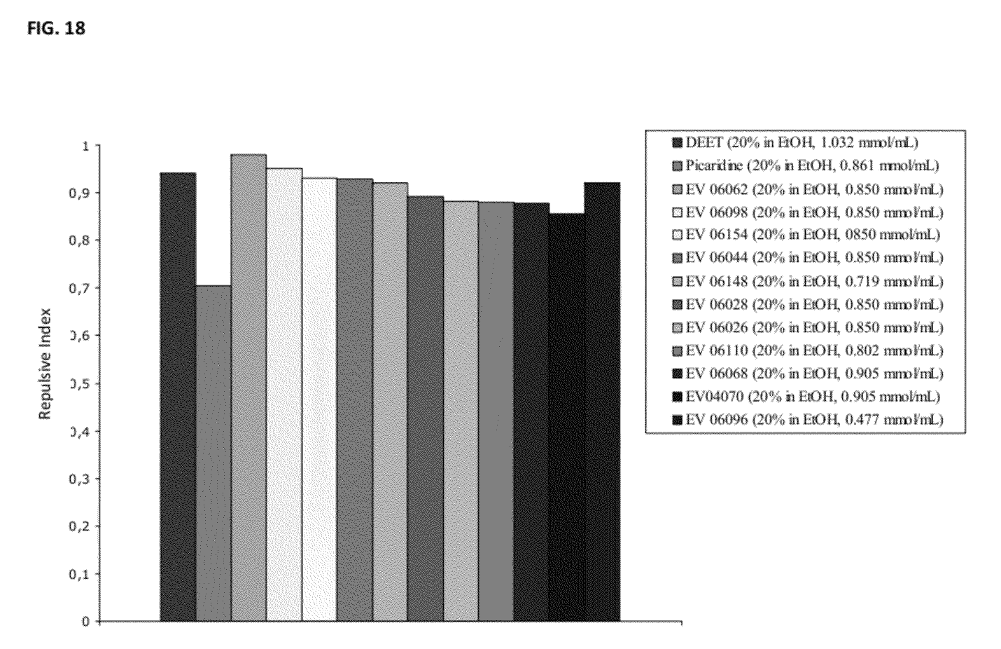

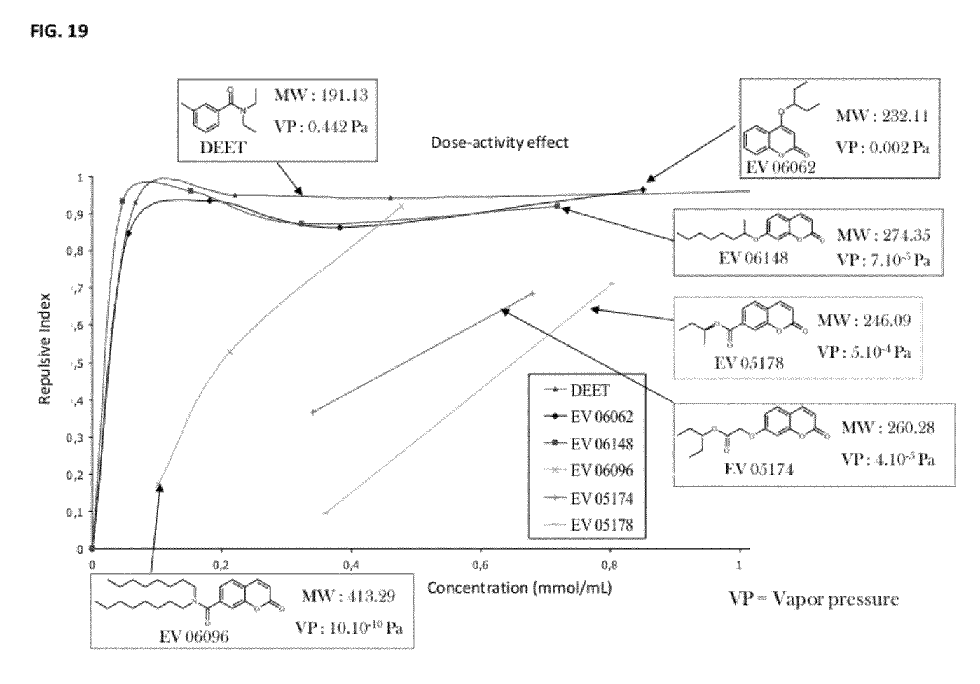

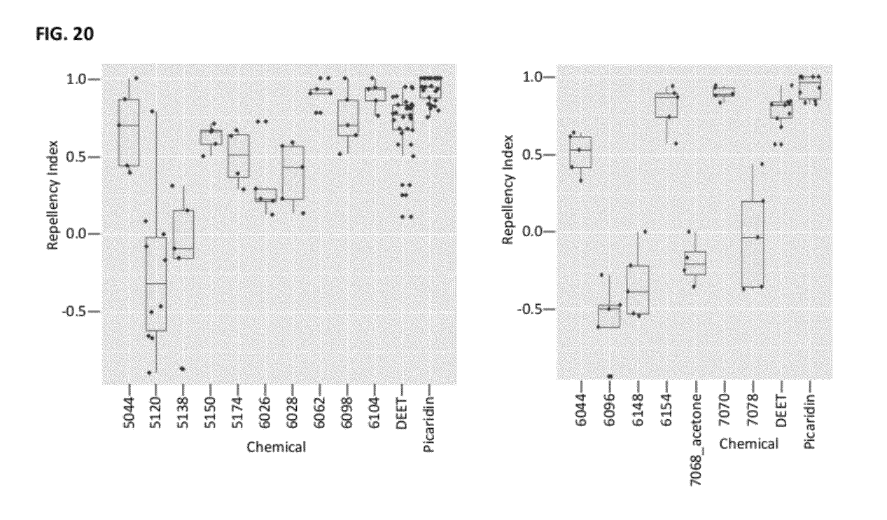

FIGS. 9-19 summarize the RI for inventive coumarin derivatives according to the instant disclosure.

### Prophetic Example

Compounds are applied to animals and surroundings to repel pests.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A compound according to formula (I) or (Ia): 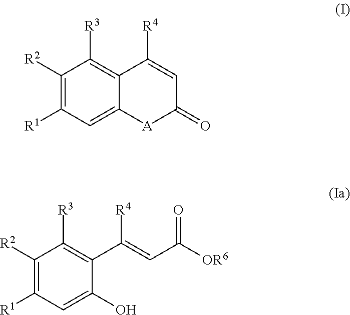
wherein
R¹, R², R³, R⁴, and R⁶ independently include H, C, OR⁵, CR⁵, OCC(═O)N(R⁵)(R⁷), CC(═O)N(R⁵)(R⁷), alkyl, aryl, aralkyl, heteroaryl, alcohols, amine, aldehyde, heterocyclyl, or Salts of amines and carboxylates;
A is O, or the ring is opened at A, thus forming compounds according to formula (Ia);
R⁵ and R⁷ are independently selected from alkyl, aryl, aralkyl, heteroaryl, alcohols, amine, aldehyde, or heterocyclyl; or R⁵ and R⁷ come together to form a C₃-C₁₀ ring, which may be aromatic, non-aromatic, and/or substituted with alkyl, aryl, aralkyl, heteroaryl, alcohols, amine, aldehyde, heterocyclyl, or combinations thereof.

2. The compound of claim 1 which is EV04016, EV04024, EV04030, EV04032, EV04036, EV04054, EV04058, EV04062, EV04070, EV04084, EV04090, EV04094, EV04114, EV04122, EV04188, EV05056, EV05084, EV05088, EV05096, EV05118, EV05120, EV05134, EV05138, EV05144, EV05162, EV05174, EV05178, EV05184, EV06018, EV06020, EV06026, EV06028, EV06036, EV06044, EV06046, EV06048, EV06056, EV06058, EV06062, EV06068, EV06072, EV06086, EV06088, EV06092, EV06094, EV06096, EV06098, EV06110, EV06128, EV06136, EV06140, EV06144, EV06148, EV06154, EV06162, EV06166, EV06178, EV06188, EV07038.

3. The compound of claim 1 wherein:
A=O;
R²=R³=R⁴ is H;
R¹ is OC(═O)OR⁵;
R⁵ is alkyl, alkenyl, or alkynyl.

4. The compound of claim 1 wherein
A=O;
R²=R³=R⁴ is H;
R¹ is OC(═O)OR⁵;
R⁵ is alkyl.

5. The compound of claim 1 wherein:
A=O;
R²=R³=R⁴ is H;
R¹ is OC(═O)R⁵;
R⁵ is alkyl, alkyl ether, CCOC(═O)C, alkyl acetate, alkenyl, alkenyl acetate, alkynyl, or alkynyl acetate.

6. The compound of claim 1 wherein:
A=O;
R²=R³=R⁴ is H;
R¹ is C(═O)OR⁵;
R⁵ is alkyl, alkenyl, or alkynyl.

7. The compound of claim 1 wherein:
A=O;
R²=R³=R⁴ is H;
R¹ is OR⁵;
R⁵ is alkyl, alkenyl, or alkynyl.

8. The compound of claim 1 wherein:
A=O;
R¹=R³=R⁴ is H;
R² is OR⁵ or CR⁵;
R⁵ is alkyl, alkenyl, or alkynyl.

9. The compound of claim 1 wherein:
A=O;
R¹=R²=R⁴ is H;
R³ is OC(═O)OR⁵;
R⁵ is alkyl, alkenyl, or alkynyl.

10. The compound of claim 1 wherein:
A=O;
R¹=R²=R⁴ is H;
R³ is OC(═O)OR⁵;
R⁵ is alkyl.

11. The compound of claim 1 wherein:
A=O;
R¹=R²=R⁴ is H;
R³ is OC(═O)R⁵;
R⁵ is alkyl, alkyl ether, CCOC(═O)C, alkyl acetate, alkenyl, alkenyl acetate, alkynyl, or alkynyl acetate.

12. The compound of claim 1 wherein:
A=O;
R¹=R²=R⁴ is H;
R³ is C(═O)OR⁵;
R⁵ is alkyl, alkenyl, or alkynyl.

13. The compound of claim 1 wherein:
A=O;
R¹=R²=R⁴ is H;
R³ is OR⁵;
R⁵ is alkyl, alkenyl, or alkynyl.

14. The compound of claim 1 wherein:
A=O
R¹=R²=R³ is H;
R⁴ is OC(═O)OR⁵;
R⁵ is alkyl, alkenyl, or alkynyl.

15. An insect/pest repellent composition comprising the compound of claim 1 or 2.

16. The composition of claim 15 that is in a form suitable for topical application to an animal.

17. The composition of claim 16 that is a cream, gel, spray, liquid or spot-on.

18. A method for repelling pests comprising the step of applying a compound or composition of any of the proceeding claims to animals or a locus.

19. The method of claim 18 wherein the animals are birds or mammals.

20. The method of claim 19 wherein the mammals are humans, equines, felines, canines, bovines, or caprines.
